(19) United States
(12) Patent Application Publication
(10) Pub. No.: US 2017/0183297 A1
(43) Pub. Date: Jun. 29, 2017

(54) OMEGA-3 ANALOGUES

(71) Applicants: The University of Sydney, The University of Sydney (AU); University of Technology, Sydney, Ultimo (AU)

(72) Inventors: Tristan Rawling, Ultimo (AU); Michael Murray, The University of Sydney (AU)

(73) Assignees: The University of Sydney, The University of Sydney (AU); The University of Technology Sydney, Ultimo (AU)

(21) Appl. No.: 15/313,486

(22) PCT Filed: May 22, 2015
(86) PCT No.: PCT/AU2015/050267

§ 371 Date: Nov. 22, 2016

(30) Foreign Application Priority Data
May 22, 2014 (AU) 2014901922

(51) Int. Cl. C07C 275/30; C07C 275/28; C07C 323/44

(52) U.S. Cl. CPC C07C 275/30; C07C 323/44; C07C 275/28

(57) ABSTRACT

The present invention relates to new omega-3 fatty acid analogues and to their use in cancer therapy, including anti-metastatic therapy.

### FIELD OF THE INVENTION

The present invention relates to new fatty acid analogues and to cancer therapy, including anti-metastatic therapy.

### BACKGROUND OF THE INVENTION

The two major classes of dietary poly-unsaturated fatty acids (PUFAs) are the omega-3 and omega-6 PUFAs, typified by eicosapentaenoic acid (EPA) and arachidonic acid (AA), respectively. These PUFAs are structurally analogous, except that EPA has an additional olefinic bond between carbons 17 and 18 that is absent in AA.

High dietary intake of omega-6 PUFAs has been linked to an increased risk for prostate and other cancers, whereas omega-3 PUFA intake decreases risk (Berquin et al. 2011). However, anticancer strategies based on altered dietary regimen are unrealistic because of low patient compliance.

In the cell, both omega-3 and omega-6 PUFAs undergo biotransformation by cytochrome P450 (CYP), lipoxygenase and cyclooxygenase enzymes, which generate parallel series of eicosanoid metabolites with distinct biological actions, and mediate most of the cellular effects of PUFAs. Cyclooxygenases give rise to prostaglandins, lipoxygenases produce leukotrienes and CYPs generate PUFA epoxides.

Four enantiomeric monoepoxides (or EETs) are formed by CYP oxidation at each of the 5,6-, 8,9-, 11,12- and 14,15-olefinic double bonds of the omega-6 PUFA AA (Chen et al. 1998). In the case of the omega-3 PUFA EPA, CYPs also epoxygenate the fifth olefinic bond at C17-18, as well as the other four double bonds.

While dietary C17,18 omega-3 PUFA epoxides are understood to provide decreased risk of cancer, they are not produced in sufficient amounts in the body to have a therapeutic effect, and their duration of action is limited by the enzyme cytosolic epoxide hydrolase (cEH), which mediates their hydration to inactive diols (Inceoglue et al. 2007).

New anti-metastatic therapies, including therapies that target various stages of metastasis, are required.

Reference to any prior art in the specification is not, and should not be taken as, an acknowledgment or any form of suggestion that this prior art forms part of the common general knowledge in Australia or any other jurisdiction or that this prior art could reasonably be expected to be ascertained, understood and regarded as relevant by a person skilled in the art.

### SUMMARY OF THE INVENTION

The present invention relates to a compound of formula (I):

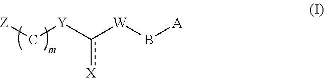

wherein

A is selected from OR¹, C(O)R¹, C(O)OR¹, C(O)NR¹R², OP(O)(OR¹)₂, C(O)OP(O)(OR)₂, P(OR¹)₃, C(O)OP(OR¹)₃, C(O)P(OR¹)₃, OS(O)(OR¹)₂, C(O)S(O)(OR¹)₂, OS(O)₂(OR¹), C(O)S(O)₂(OR¹), OSR¹, C(O)SR, OSR¹R², C(O)SR¹R², cycloalkyl, heterocycloalkyl and heteroaryl;

B is a hydrocarbon chain containing from 7 to 25 carbon atoms, wherein the hydrocarbon chain is saturated or unsaturated, branched or unbranched, and includes one or more heteroatoms selected from O, N and S;

W and Y are selected from CH₂, O and NR¹, wherein W may form a 5- or 6-membered cycloalkyl or heterocycloalkyl ring with X and B;

X is selected from CH₂, O, NR¹ and S;

C is CH₂;

m is 0, 1 or 2;

Z is selected from alkyl, heteroalkyl, alkenyl, alkynyl, cycloalkyl, heterocycloalkyl, aryl and heteroaryl, which groups are optionally substituted,

wherein R¹ and R² are independently selected from H, OH, alkyl, heteroalkyl, alkenyl, alkynyl, cycloalkyl, heterocycloalkyl, alkylcycloalkyl, heteroalkylcycloalkyl, aryl, heteroaryl, aralkyl and heteroaralkyl, which groups are optionally substituted,

or a pharmaceutically acceptable salt, solvate or hydrate thereof.

The invention also relates to compositions including the above described compounds, and to uses of the compounds and compositions for treating proliferative disease, for inducing apoptosis and/or for inhibiting proliferation or metastasis.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: MTT reduction in breast cancer cell lines treated with CP01.

FIG. 2: ATP formation in breast cancer cell lines treated with CP01.

FIG. 3: JC-1 and caspase 3/7 activity for mitochondrial integrity and apoptosis—(left) Caspase 3/7 activation in MDA-MB-231 cells treated with CP01 (10 μM, 48 h) and (right) JC-1 red/green fluorescence ratio in MDA-MB-231 breast cancer cells treated with CP01 (1-20 μM, 4 h).

FIG. 4: Assays of the migration of MDA-MB-231 cells out of matrigel droplets after treatment with (A) CP02 (B) CP04 and (C) CP06.

FIG. 5: Body weight growth in control and treated groups (CP01).

FIG. 6: Body weight growth in control and treated groups (CP03).

FIG. 7: Effects of CP01 treatment on tumour growth (by volume).

FIG. 8: Effects of CP01 treatment on final tumour weight.

FIG. 9: Effects of CP03 treatment on tumour growth (by volume).

FIG. 10: Effects of CP03 treatment on final tumour weight.

FIG. 11: Effects of lower doses of CP01 on breast tumour growth.

FIG. 12: Effects of lower doses of CP02 on breast tumour growth.

FIG. 13: Effects of lower doses of CP05 on breast tumour growth.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Compounds are generally described herein using standard nomenclature. For compounds having asymmetric centres, it will be understood that, unless otherwise specified, all of the optical isomers and mixtures thereof are encompassed. Compounds with two or more asymmetric elements can also be present as mixtures of diastereomers. In addition, compounds with carbon-carbon double bonds may occur in Z and E forms, with all isomeric forms of the compounds being included in the present invention unless otherwise specified. Where a compound exists in various tautomeric forms, a recited compound is not limited to any one specific tautomer, but rather is intended to encompass all tautomeric forms. Recited compounds are further intended to encompass compounds in which one or more atoms are replaced with an isotope, i.e., an atom having the same atomic number but a different mass number. By way of general example, and without limitation, isotopes of hydrogen include tritium and deuterium and isotopes of carbon include ¹¹C, ¹³C, and ¹⁴C.

Compounds according to the formula provided herein, which have one or more stereogenic centres, have an enantiomeric excess of at least 50%. For example, such compounds may have an enantiomeric excess of at least 60%, 70%, 80%, 85%, 90%, 95%, or 98%. Some embodiments of the compounds have an enantiomeric excess of at least 99%. It will be apparent that single enantiomers (optically active forms) can be obtained by asymmetric synthesis, synthesis from optically pure precursors, biosynthesis (for example, using modified CYP102 such as CYP BM-3) or by resolution of the racemates, for example, enzymatic resolution or resolution by conventional methods such as crystallization in the presence of a resolving agent, or chromatography, using, for example, a chiral HPLC column.

Certain compounds are described herein using a general formula that includes variables such as R¹, A, B, X, Y and Z. Unless otherwise specified, each variable within such a formula is defined independently of any other variable, and any variable that occurs more than one time in a formula is defined independently at each occurrence. Therefore, for example, if a group is shown to be substituted with 0, 1 or 2 R*, the group may be unsubstituted or substituted with up to two R* groups and R* at each occurrence is selected independently from the definition of R*. Also, combinations of substituents and/or variables are permissible only if such combinations result in stable compounds, i.e., compounds that can be isolated, characterized and tested for biological activity.

A “pharmaceutically acceptable salt” of a compound disclosed herein is an acid or base salt that is generally considered in the art to be suitable for use in contact with the tissues of human beings or animals without excessive toxicity or carcinogenicity, and preferably without irritation, allergic response, or other problem or complication. Such salts include mineral and organic acid salts of basic residues such as amines, as well as alkali or organic salts of acidic residues such as carboxylic acids.

Suitable pharmaceutically acceptable salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzenesulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic (such as acetic, HOOC—(CH₂)ₙ—COOH where n is any integer from 0 to 6, i.e. 0, 1, 2, 3, 4, 5 or 6), and the like. Similarly, pharmaceutically acceptable cations include, but are not limited to sodium, potassium, calcium, aluminum, lithium and ammonium. A person skilled in the art will recognize further pharmaceutically acceptable salts for the compounds provided herein. In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in water or in an organic solvent, or in a mixture of the two. Generally, the use of nonaqueous media, such as ether, ethyl acetate, ethanol, isopropanol or acetonitrile, is preferred.

It will be apparent that each compound of formula (I) may, but need not, be present as a hydrate, solvate or non-covalent complex. In addition, the various crystal forms and polymorphs are within the scope of the present invention, as are prodrugs of the compounds of formula (I) provided herein.

A “prodrug” is a compound that may not fully satisfy the structural requirements of the compounds provided herein, but is modified in vivo, following administration to a subject or patient, to produce a compound of formula (I) provided herein. For example, a prodrug may be an acylated derivative of a compound as provided herein. Prodrugs include compounds wherein hydroxy, carboxy, amine or sulfhydryl groups are bonded to any group that, when administered to a mammalian subject, cleaves to form a free hydroxy, carboxy, amino, or sulfhydryl group, respectively. Examples of prodrugs include, but are not limited to, acetate, formate, phosphate and benzoate derivatives of alcohol and amine functional groups within the compounds provided herein. Prodrugs of the compounds provided herein may be prepared by modifying functional groups present in the compounds in such a way that the modifications are cleaved in vivo to generate the parent compounds.

A “substituent” as used herein, refers to a molecular moiety that is covalently bonded to an atom within a molecule of interest. For example, a “ring substituent” may be a moiety such as a halogen, alkyl group, heteroalkyl group, haloalkyl group or other substituent described herein that is covalently bonded to an atom, preferably a carbon or nitrogen atom, that is a ring member. The term “substituted,” as used herein, means that any one or more hydrogens on the designated atom is replaced with a selection from the indicated substituents, provided that the designated atom's normal valence is not exceeded, and that the substitution results in a stable compound, i.e., a compound that can be isolated, characterized and tested for biological activity. When a substituent is oxo, i.e., ═O, then two hydrogens on the atom are replaced. An oxo group that is a substituent of an aromatic carbon atom results in a conversion of —CH— to —C(═O)— and a loss of aromaticity. For example a pyridyl group substituted by oxo is a pyridone. Examples of suitable substituents are alkyl (including haloalkyl e.g. CF₃), heteroalkyl, halogen (for example, fluorine, chlorine, bromine or iodine atoms), C(O)OR¹ (e.g. C(O)OH), C(O)R¹ (e.g. C(O)H), OH, ═O, SH, SO₃H, NH₂, NH-alkyl, NR¹₃⁺ (e.g. N(CH₃)₃⁺), ═NH, N₃ and NO₂ groups.

The term “alkyl” refers to a saturated, straight-chain or branched hydrocarbon group that contains from 1 to 20 carbon atoms, preferably from 1 to 10 carbon atoms, for example a n-octyl group, especially from 1 to 6, i.e. 1, 2, 3, 4, 5, or 6, carbon atoms. Specific examples of alkyl groups are methyl, ethyl, propyl, iso-propyl, n-butyl, iso-butyl, sec-butyl, tert-butyl, n-pentyl, iso-pentyl, n-hexyl and 2,2-dimethylbutyl.

The term “heteroalkyl” refers to an alkyl group as defined above that contains one or more heteroatoms selected from oxygen, nitrogen and sulphur (especially oxygen and nitrogen). Specific examples of heteroalkyl groups are methoxy, trifluoromethoxy, ethoxy, n-propyloxy, iso-propyloxy, butoxy, tert-butyloxy, methoxymethyl, ethoxymethyl, —CH₂CH₂OH, —CH₂OH, methoxyethyl, 1-methoxyethyl, 1-ethoxyethyl, 2-methoxyethyl or 2-ethoxyethyl, methylamino, ethylamino, propylamino, iso-propylamino, dimethylamino, diethylamino, iso-propyl-ethylamino, methylamino methyl, ethylamino methyl, di-iso-propylamino ethyl, methylthio, ethylthio, iso-propylthio, enol ether, dimethylamino methyl, dimethylamino ethyl, acetyl, propionyl, butyryloxy, acetyloxy, methoxycarbonyl, ethoxycarbonyl, propionyloxy, acetylamino, propionylamino, carboxymethyl, carboxyethyl, carboxypropyl, N-ethyl-N-methylcarbamoyl and N-methylcarbamoyl. Further examples of heteroalkyl groups are nitrile, iso-nitrile, cyanate, thiocyanate, iso-cyanate, iso-thiocyanate and alkylnitrile groups.

The term “alkenyl” refers to an at least partially unsaturated, straight-chain or branched hydrocarbon group that contains from 2 to 20 carbon atoms, preferably from 2 to 10 carbon atoms, especially from 2 to 6, i.e. 2, 3, 4, 5 or 6, carbon atoms. Specific examples of alkenyl groups are ethenyl (vinyl), propenyl (allyl), iso-propenyl, butenyl, ethinyl, propinyl, butinyl, acetylenyl, propargyl, iso-prenyl and hex-2-enyl group.

Preferably, alkenyl groups have one or two double bond(s).

The term “alkynyl” refers to an at least partially unsaturated, straight-chain or branched hydrocarbon group that contains from 2 to 20 carbon atoms, preferably from 2 to 10 carbon atoms, especially from 2 to 6, i.e. 2, 3, 4, 5 or 6, carbon atoms. Specific examples of alkynyl groups are ethynyl, propynyl, butynyl, acetylenyl and propargyl groups. Preferably, alkynyl groups have one or two (especially preferably one) triple bond(s).

The term “cycloalkyl” refers to a saturated or partially unsaturated (for example, a cycloalkenyl group) cyclic group that contains one or more rings (preferably 1 or 2), and contains from 3 to 14 ring carbon atoms, preferably from 3 to 10 (especially 3, 4, 5, 6 or 7) ring carbon atoms. Specific examples of cycloalkyl groups are a cyclopropyl, cyclobutyl, cyclopentyl, spiro[4,5]decanyl, norbornyl, cyclohexyl, cyclopentenyl, cyclohexadienyl, decalinyl, bicyclo[4.3.0]nonyl, tetraline, adamantane (i.e. tricycle[3.3.1.1^{3,7}]decane), cyclopentylcyclohexyl and cyclohex-2-enyl.

The term “heterocycloalkyl” refers to a cycloalkyl group as defined above in which one or more (preferably 1, 2 or 3) ring carbon atoms, each independently, have been replaced by an oxygen, nitrogen, silicon, selenium, phosphorus or sulfur atom (preferably by an oxygen, sulfur or nitrogen atom). A heterocycloalkyl group has preferably 1 or 2 rings containing from 3 to 10 (especially 3, 4, 5, 6 or 7) ring atoms (preferably selected from C, O, N and S). Specific examples are piperidyl, prolinyl, imidazolidinyl, piperazinyl, morpholinyl, urotropinyl, pyrrolidinyl, tetrahydrothiophenyl, tetrahydropyranyl, tetrahydrofuryl and 2-pyrazolinyl group and also lactames, lactones, cyclic imides and cyclic anhydrides.

The term “alkylcycloalkyl” refers to a group that contains both cycloalkyl and also alkyl, alkenyl or alkynyl groups in accordance with the above definitions, for example alkylcycloalkyl, cycloalkylalkyl, alkylcycloalkenyl, alkenylcycloalkyl and alkynylcycloalkyl groups. An alkylcycloalkyl group preferably contains a cycloalkyl group that contains one or two ring systems having from 3 to 10 (especially 3, 4, 5, 6 or 7) ring carbon atoms, and one alkyl, alkenyl or alkynyl group having 1 or 2 to 6 carbon atoms. The alkyl, alkenyl or alkynyl groups may form a bi- or tri-cyclic ring system with the cycloalkyl group, and may be the means by which the cycloalkyl group is joined to the compound of formula (I).

The term “heteroalkylcycloalkyl” refers to alkylcycloalkyl groups as defined above in which one or more, preferably 1, 2 or 3, carbon atoms have been replaced independently of each other by an oxygen, nitrogen, silicon, selenium, phosphorus or sulfur atom (preferably by an oxygen, sulfur or nitrogen atom). A heteroalkylcycloalkyl group preferably contains 1 or 2 ring systems having from 3 to 10 (especially 3, 4, 5, 6 or 7) ring atoms, and one or two alkyl, alkenyl, alkynyl or heteroalkyl groups having from 1 or 2 to 6 carbon atoms. Examples of such groups are alkylheterocycloalkyl, alkylheterocycloalkenyl, alkenylheterocycloalkyl, alkynylheterocycloalkyl, heteroalkylcycloalkyl, heteroalkyl-heterocycloalkyl and heteroalkylheterocycloalkenyl, the cyclic groups being saturated or mono-, di- or tri-unsaturated.

The term “aryl” refers to an aromatic group that contains one or more rings containing from 6 to 14 ring carbon atoms, preferably from 6 to 10 (especially 6) ring carbon atoms. Examples are phenyl, naphthyl and biphenyl groups.

The term “heteroaryl” refers to an aromatic group that contains one or more rings containing from 5 to 14 ring atoms, preferably from 5 to 10 (especially 5 or 6) ring atoms, and contains one or more (preferably 1, 2, 3 or 4) oxygen, nitrogen, phosphorus or sulfur ring atoms (preferably O, S or N). Examples are pyridyl (for example, 4-pyridyl), imidazolyl (for example, 2-imidazolyl), phenylpyrrolyl (for example, 3-phenylpyrrolyl), thiazolyl, iso-thiazolyl, 1,2,3-triazolyl, 1,2,4-triazolyl, oxadiazolyl, thiadiazolyl, indolyl, indazolyl, tetrazolyl, pyrazinyl, pyrimidinyl, pyridazinyl, oxazolyl, isoxazolyl, triazolyl, tetrazolyl, isoxazolyl, indazolyl, indolyl, benzimidazolyl, benzoxazolyl, benzisoxazolyl, benzthiazolyl, pyridazinyl, quinolinyl, isoquinolinyl, pyrrolyl, purinyl, carbazolyl, acridinyl, pyrimidyl, 2,3′-bifuryl, pyrazolyl (for example, 3-pyrazolyl) and iso-quinolinyl groups.

The term “aralkyl” refers to a group containing both aryl and also alkyl, alkenyl, alkynyl and/or cycloalkyl groups in accordance with the above definitions, such as, for example, an arylalkyl, arylalkenyl, arylalkynyl, arylcycloalkyl, aryl-cycloalkenyl, alkylarylcycloalkyl and alkylarylcycloalkenyl group. The alkyl, alkenyl or alkynyl groups may provide the means by which the alkyl group is joined to the compound of formula (I). Specific examples of aralkyls are 1H-indene, tetraline, dihydronaphthalene, indanone, phenylcyclopentyl, cyclohexylphenyl, fluorene and indane. An aralkyl group preferably contains one or two aromatic ring systems (1 or 2 rings) containing from 6 to 10 carbon atoms and one alkyl, alkenyl and/or alkynyl group containing from 1 or 2 to 6 carbon atoms and/or a cycloalkyl group containing 5 or 6 ring carbon atoms.

The term “heteroaralkyl” refers to an aralkyl group as defined above in which one or more (preferably 1, 2, 3 or 4) carbon atoms, each independently, have been replaced by an oxygen, nitrogen, silicon, selenium, phosphorus, boron or sulfur atom (preferably oxygen, sulfur or nitrogen). That is, a group containing aryl or heteroaryl, respectively, and also alkyl, alkenyl, alkynyl and/or heteroalkyl and/or cycloalkyl and/or heterocycloalkyl groups in accordance with the above definitions. A heteroaralkyl group preferably contains one or two aromatic ring systems (1 or 2 rings) containing from 5 or 6 to 10 ring carbon atoms and one alkyl, alkenyl and/or alkynyl group containing 1 or 2 to 6 carbon atoms and/or a cycloalkyl group containing 5 or 6 ring carbon atoms, wherein 1, 2, 3 or 4 of these carbon atoms have been replaced by oxygen, sulfur or nitrogen atoms. The alkyl, alkenyl or alkynyl group may provide the means by which the alkyl group is joined to the compound of formula (I).

Examples are arylheteroalkyl, arylheterocycloalkyl, arylheterocycloalkenyl, arylalkylheterocycloalkyl, arylalkenyl-heterocycloalkyl, arylalkynylheterocycloalkyl, arylalkyl-heterocycloalkenyl, heteroarylalkyl, heteroarylalkenyl, heteroarylalkynyl, heteroarylheteroalkyl, heteroaryl-cycloalkyl, heteroarylcycloalkenyl, heteroarylhetero-cycloalkyl, heteroarylheterocycloalkenyl, heteroarylalkyl-cycloalkyl, heteroarylalkylheterocycloalkenyl, heteroaryl-heteroalkylcycloalkyl, heteroarylheteroalkylcycloalkenyl and heteroarylheteroalkylheterocycloalkyl groups, the cyclic groups being saturated or mono-, di- or tri-unsaturated. Specific examples are tetrahydroisoquinolinyl and benzoyl.

The expression “halogen” or “halogen atom” as used herein means fluorine, chlorine, bromine, or iodine.

The term “optionally substituted” refers to a group in which one, two, three or more hydrogen atoms have been replaced independently of each other by halogen (for example, fluorine, chlorine, bromine or iodine atoms) and/or by C(O)OR¹ (e.g. C(O)OH), C(O)R¹ (e.g. C(O)H), OH, ═O, SH, ═S, SO₃H, NH₂, NH-alkyl, NR¹₃⁺ (e.g. N(CH₃)₃⁺), ═NH, N₃ or NO₂ groups. This expression also refers to a group that is substituted by one, two, three or more alkyl, alkenyl, alkynyl, heteroalkyl, cycloalkyl, heterocycloalkyl, alkylcycloalkyl, heteroalkylcycloalkyl, aryl, heteroaryl, aralkyl or heteroaralkyl groups. These groups may themselves be substituted. For example, an alkyl group substituent may be substituted by one or more halogen atoms (i.e. may be a haloalkyl group). The term “haloalkyl” refers to an alkyl group (as defined above) that is substituted by one or more halogen atoms (as also defined above). Specific examples of haloalkyl groups are trifluoromethyl, dichloroethyl, dichloromethyl and iodoethyl.

As used herein a wording defining the limits of a range of length such as, for example, “from 1 to 5” means any integer from 1 to 5, i. e. 1, 2, 3, 4 and 5. In other words, any range defined by two integers explicitly mentioned is meant to comprise and disclose any integer defining said limits and any integer comprised in said range.

In one embodiment of the compound of formula (I), A is selected from C(O)R¹, C(O)OR¹, C(O)NR¹R², OP(O)(OR¹)₂, C(O)OP(O)(OR¹)₂, P(OR¹)₃, C(O)OP(OR¹)₃, C(O)P(OR¹)₃, OS(O)(OR¹)₂, C(O)S(O)(OR¹)₂, OS(O)₂(OR¹), C(O)S(O)₂(OR¹), OSR¹, C(O)SR¹, OSR¹R² and C(O)SR¹R². For example, A may be C(O)R¹, C(O)OR¹ or C(O)NR¹R². In one embodiment R¹ is alkyl (e.g. C₁-C₆ alkyl). For example, R¹ may be methyl, ethyl, propyl, butyl, pentyl or hexyl, which groups may be branched or unbranched. In one embodiment, A is C(O)OR¹ (e.g. C(O)OH, C(O)OCH₃ or C(O)OCH₂CH₃).

In another embodiment, B is a hydrocarbon chain containing from 10 to 15 carbon atoms (e.g. 10, 11, 12, 13, 14 or 15 carbon atoms). In one embodiment, the hydrocarbon chain is saturated. In another embodiment, the hydrocarbon chain is unbranched.

In one embodiment, the heteroatom of the hydrocarbon chain is a S atom. In another embodiment, the hydrocarbon chain includes two or more S atoms. In one embodiment, the heteroatom of the hydrocarbon chain is an O atom. In another embodiment, the hydrocarbon chain includes two or more O atoms. Where more than one heteroatom is present, the heteroatoms may be the same heteroatom (e.g. all the heteroatoms are S, N or O), or they may be a mixture of different heteroatoms (e.g. one O atom and one S atom).

In one embodiment, the heteroatom(s) are in any position along the hydrocarbon chain. In another embodiment, the heteroatom(s) are at the 3-, 4-, 5-, 6-, 7-, 8-, 9-, 10-, 11-, 12- and/or 13-positions along the hydrocarbon chain. Preferably, the heteroatom(s) are at the 3- and/or 13-positions along the hydrocarbon chain.

In one embodiment, W and Y are NR¹ (R¹ may be, for example, H or alkyl). In one embodiment, R¹ is alkyl (e.g. C1-C₆ alkyl). For example, R¹ may be methyl, ethyl, propyl, butyl, pentyl or hexyl, which groups may be branched or unbranched.

In one embodiment, X is O and the bond between X and the carbon atom to which X is attached is a double bond.

In one embodiment, m is 0.

Preferred compounds of formula (I) are those where Z is a cycloalkyl group, an aryl group or a branched alkyl group (for example, a tert-butyl group). Preferably, the cycloalkyl group is a cyclopentyl, cyclohexyl or cycloheptyl group, and the aryl group is a 5- or 6-membered aryl ring (e.g. a phenyl group). Preferably, Z is an aryl group. The aryl group may be a phenyl group. In one embodiment, the phenyl group is substituted. In one embodiment, Z is substituted by either a halogen (for example, fluorine, chlorine or iodine) or an alkyl group (for example, methyl, ethyl or propyl).

In another embodiment, Z is substituted by one or more halogens, one or more alkyl groups, one or more heteroalkyl groups, or combinations thereof. In one embodiment, Z is substituted by two halogens. Z may be substituted by a halogen and an alkyl group, and the alkyl group may be a substituted alkyl group (e.g. substituted by two or more halogen atoms). In one embodiment, the substituted alkyl group is CF₃. Z may also be substituted by a heteroalkyl group (e.g. a methoxy or ethoxy group). In one embodiment, Z is substituted by an alkyl group (e.g. methyl, ethyl or propyl). In one embodiment, Z is substituted by an electron-withdrawing group (e.g. CN, C(O)OR¹ (e.g. C(O)O or C(O)) alkyl), C(O)R¹ (e.g. C(O)H or C(O) alkyl), CCl₃, NO₂, CF₃, SO₃H, NR¹₃⁺ (e.g. N(alkyl)₃⁺, such as N(CH₃)₃⁺).

Specific examples of the compounds of the present invention are given in Table 1, below.

TABLE 1
Examples of compounds of the present invention

| Compound | Structure |
|---|---|
| CP01 | 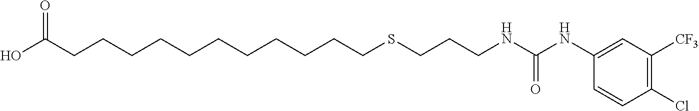 [SMILES: O=C(O)CCCCCCCCCCCSCCCNC(=O)Nc1ccc(Cl)c(C(F)(F)F)c1] |
| CP02 | 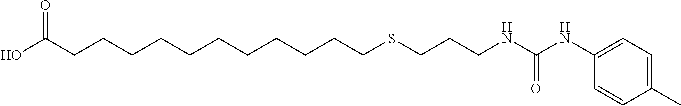 [SMILES: Cc1ccc(NC(=O)NCCCSCCCCCCCCCCCC(=O)O)cc1] |
| CP03 | 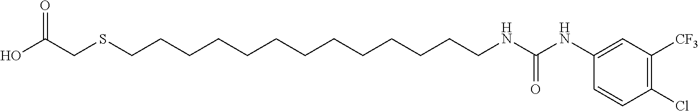 [SMILES: O=C(O)CSCCCCCCCCCCCCCNC(=O)Nc1ccc(Cl)c(C(F)(F)F)c1] |
| CP04 | 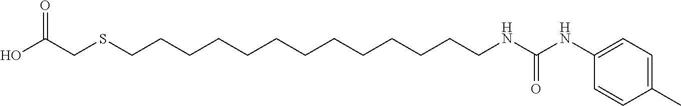 [SMILES: Cc1ccc(NC(=O)NCCCCCCCCCCCCCSCC(=O)O)cc1] |
| CP05 | 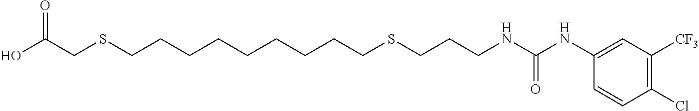 [SMILES: O=C(O)CSCCCCCCCCCSCCCNC(=O)Nc1ccc(Cl)c(C(F)(F)F)c1] |
| CP06 | 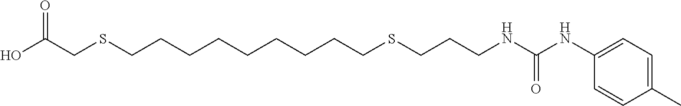 [SMILES: Cc1ccc(NC(=O)NCCCSCCCCCCCCCSCC(=O)O)cc1] |
| CP07 | 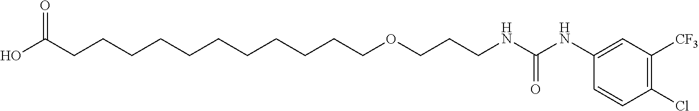 [SMILES: O=C(O)CCCCCCCCCCCOCCCNC(=O)Nc1ccc(Cl)c(C(F)(F)F)c1] |
| CP08 | 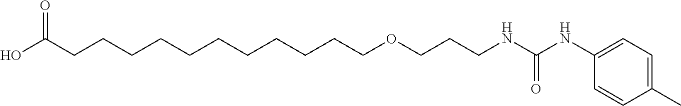 [SMILES: Cc1ccc(NC(=O)NCCCOCCCCCCCCCCCC(=O)O)cc1] |
| CP09 | 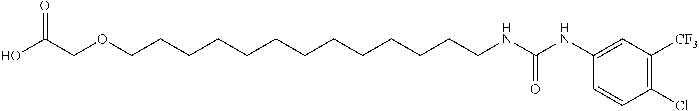 [SMILES: O=C(O)COCCCCCCCCCCCCCNC(=O)Nc1ccc(Cl)c(C(F)(F)F)c1] |
| CP10 | 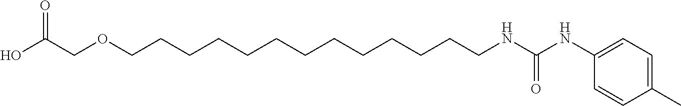 [SMILES: Cc1ccc(NC(=O)NCCCCCCCCCCCCCOCC(=O)O)cc1] |
| CP11 | 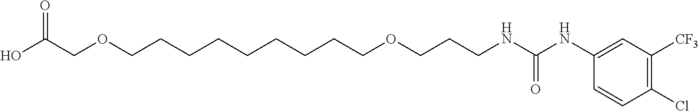 [SMILES: O=C(O)COCCCCCCCCCOCCCNC(=O)Nc1ccc(Cl)c(C(F)(F)F)c1] |
| CP12 | 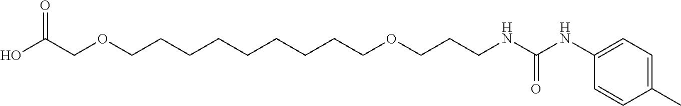 [SMILES: Cc1ccc(NC(=O)NCCCOCCCCCCCCCOCC(=O)O)cc1] |

In one embodiment, the compound of formula (I) is selected from the group consisting of compounds 1 to 12 (i.e. CP01 to CP12) from Table 1 above.

In one embodiment, the compound of formula (I) is selected from the group consisting of compounds 1 to 6 (i.e. CP01 to CP06) from Table 1 above.

The compounds of the present invention can be synthesised by any suitable method known to a person skilled in the art. General syntheses are given below in Schemes 1 to 8.

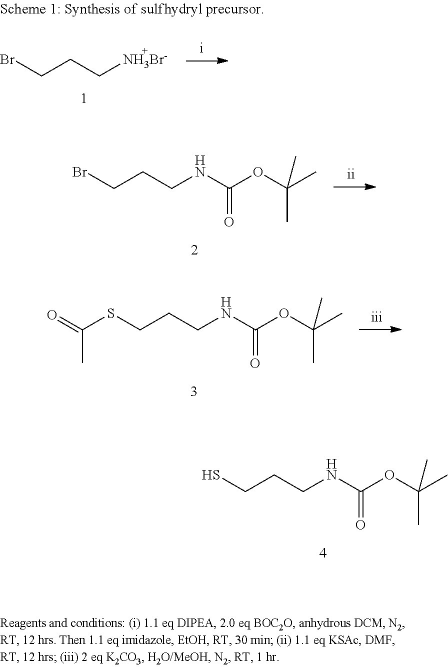

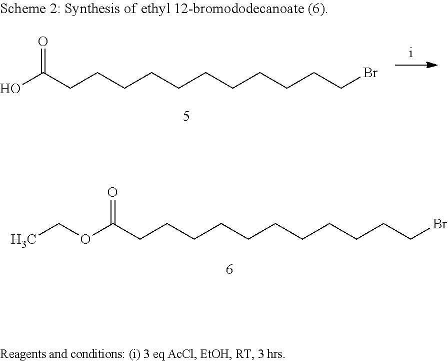 [SMILES: O=C(O)CCCCCCCCCCCBr ; CCOC(=O)CCCCCCCCCCCBr]

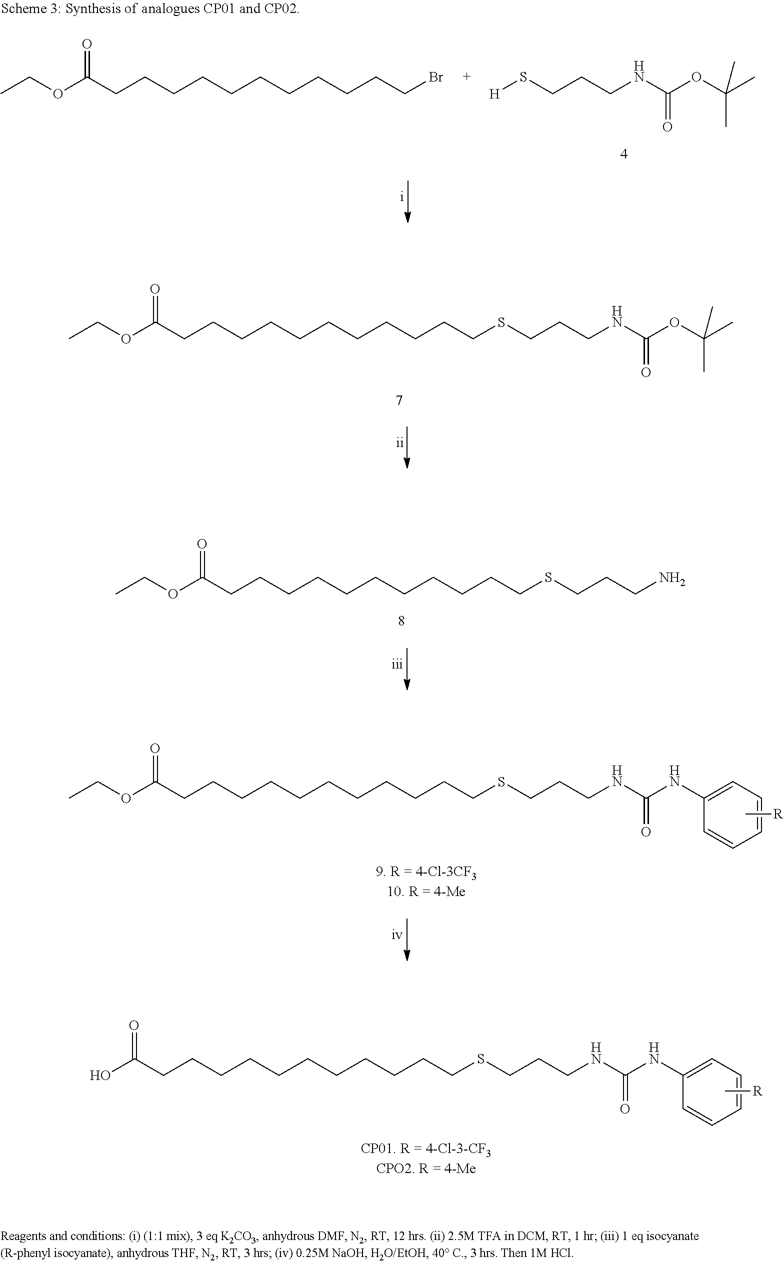

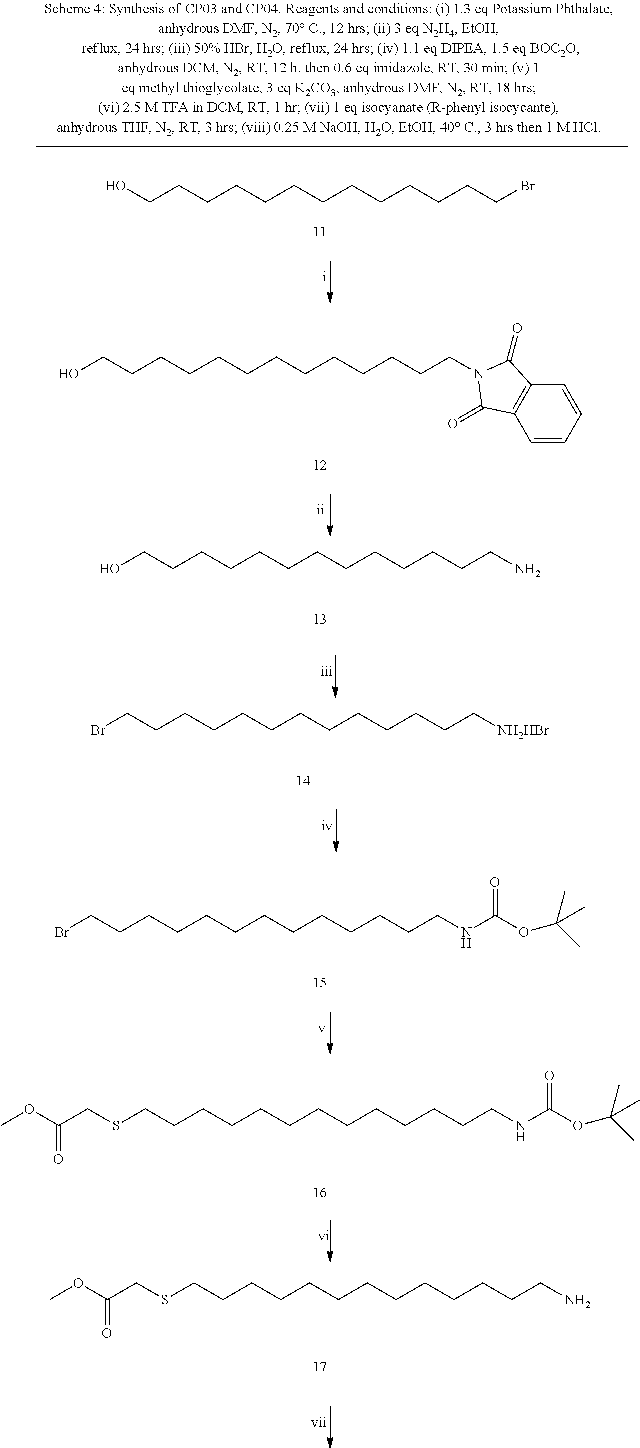

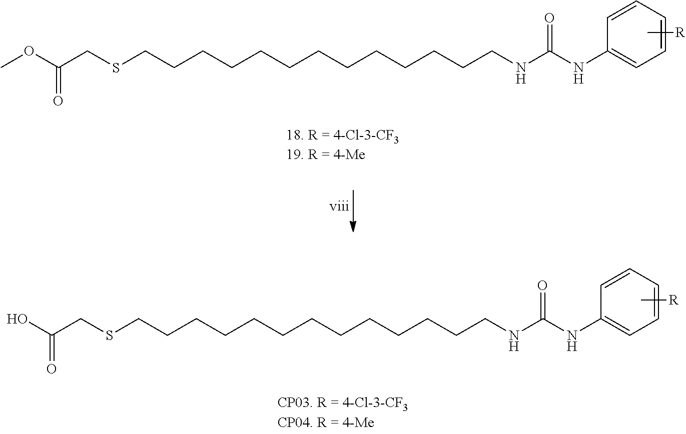

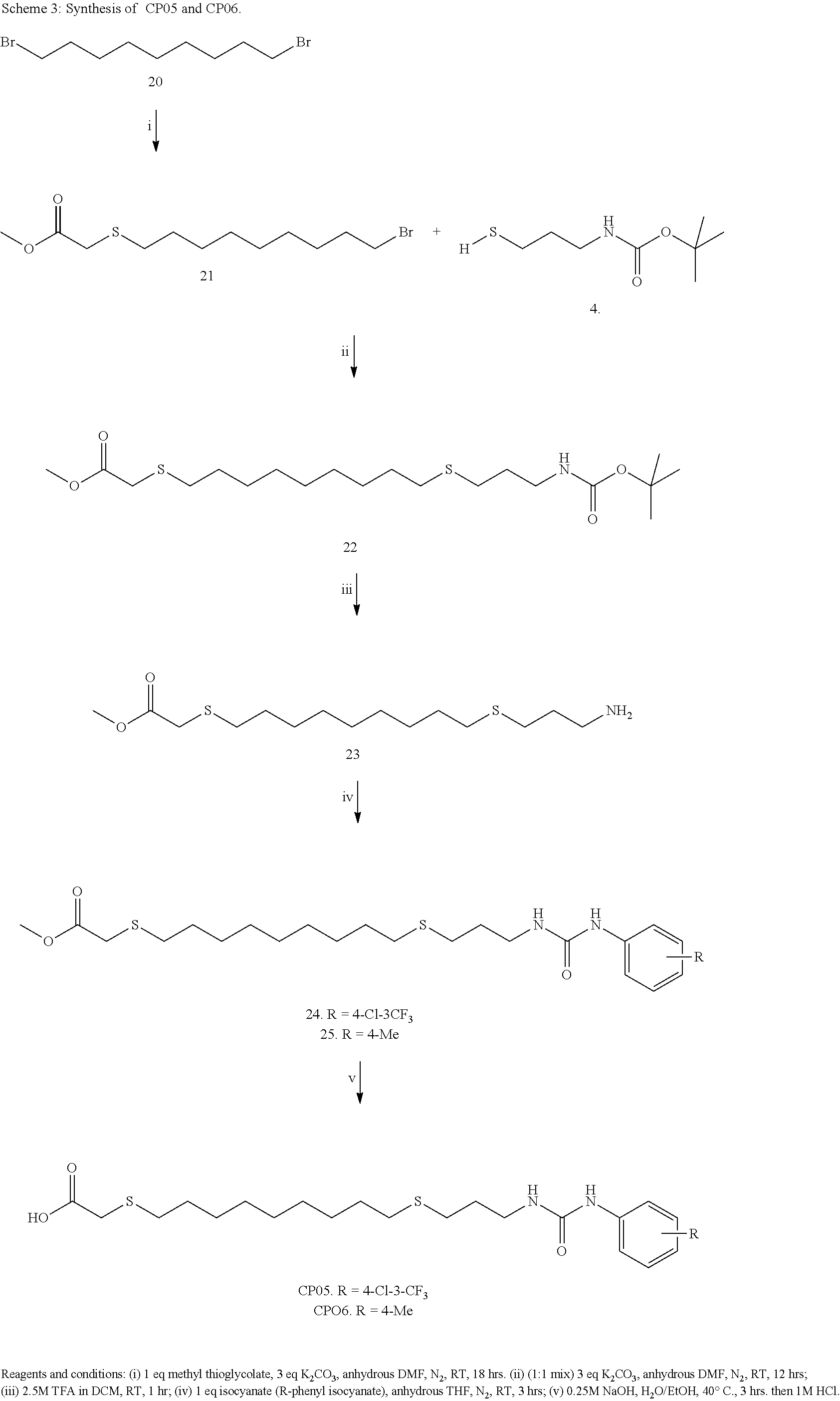

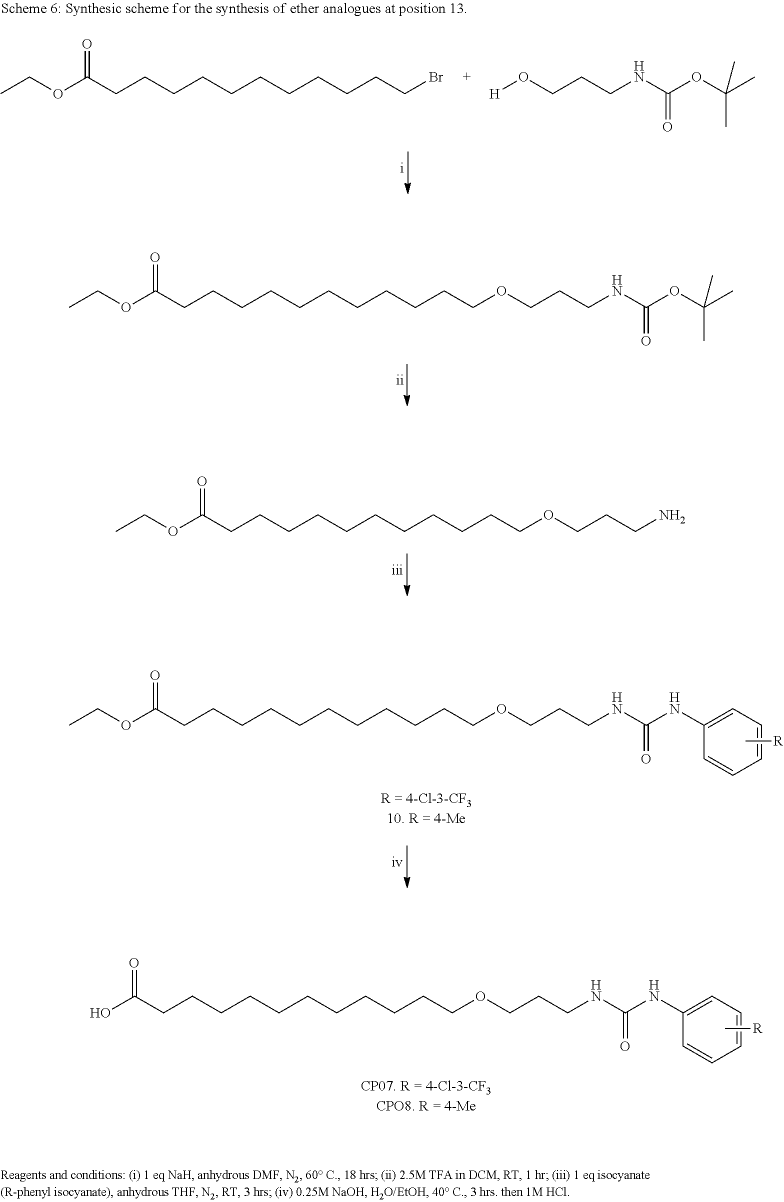

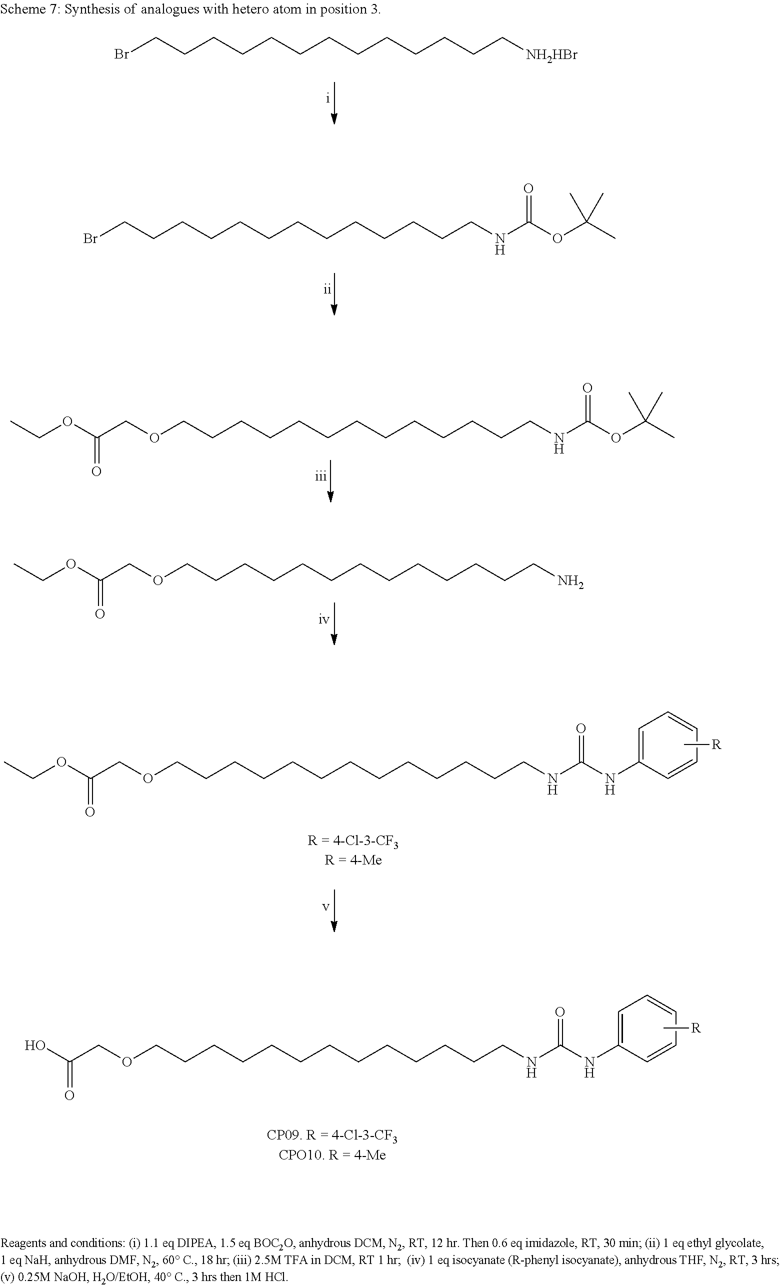

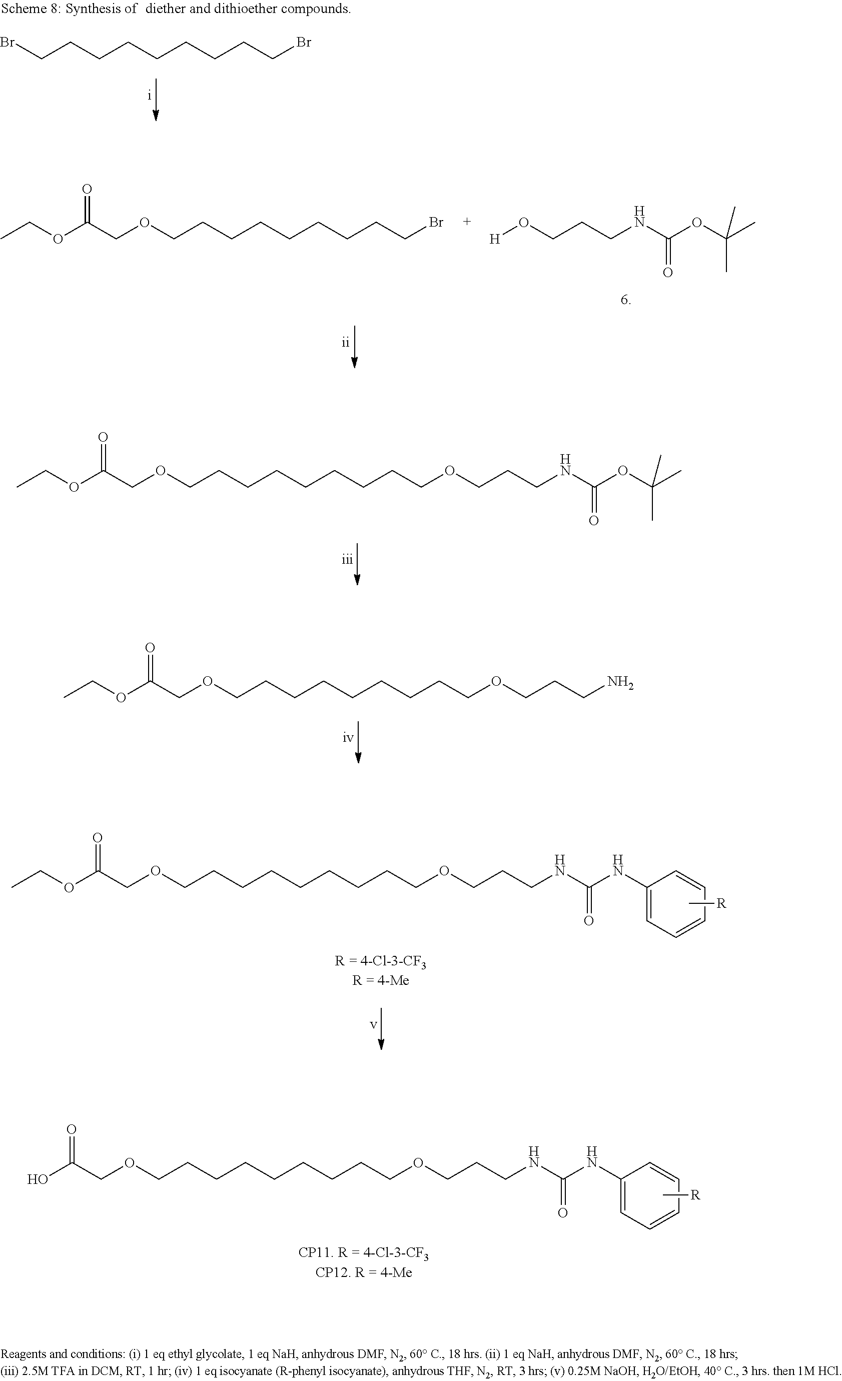

A person skilled in the art will understand that if analogues bearing, for example, branched alkyl, aryl or cycloalkyl groups are desired, the corresponding starting materials (for example, cycloalkyl- or aryl-isocyanates, or branched alkyl-isocyanates) will need to be used.

The compounds of the present invention may exhibit high anti-proliferative and anti-metastatic activities and in particular, high activity in cancer cell killing. Specifically, in the examples herein, specific compounds are shown to inhibit proliferation, to induce markers of apoptosis and/or to inhibit cell migration. The present inventors have found that compounds containing an aryl group (e.g. where Z is a phenyl group), and where the aryl group is further substituted by one or more electron-withdrawing groups (such as NO₂, CF₃ and/or SO₃H), and where the long methylene chain (i.e. the hydrocarbon chain “B”) contains heteroatoms such as S or O, are particularly effective at inducing apoptosis in primary cancer cells.

Cells undergoing proliferation may be generally classified as cells in the G₁, S, G₂ or M phase of the cell cycle. In certain embodiments, a compound of the invention may inhibit a cell from entering or from leaving any one of these phases, for example by inducing apoptosis or cell death.

In certain embodiments, the compounds of the present invention may be resistant to cEH-dependent hydration, but still have the beneficial anti-proliferative activity of omega-3 17,18-epoxy-EPA.

The therapeutic use of compounds of formula (I), their pharmaceutically acceptable salts, solvates or hydrates and also formulations and pharmaceutical compositions (including mixtures of the compounds of formula (I)) are within the scope of the present invention. Accordingly, the present invention also relates to pharmaceutical compositions including a therapeutically effective amount of the compounds of formula (I), or its pharmaceutically acceptable salt, solvate or hydrate thereof, and one or more pharmaceutically acceptable excipients.

The pharmaceutical compositions according to the present invention include at least one compound of formula (I) and, optionally, one or more carrier substances, for example, cyclodextrins such as hydroxypropyl β-cyclodextrin, micelles or liposomes, excipients and/or adjuvants. Pharmaceutical compositions may additionally include, for example, one or more of water, buffers (for example, neutral buffered saline or phosphate buffered saline), ethanol, mineral oil, vegetable oil, dimethylsulfoxide, carbohydrates (for example, glucose, mannose, sucrose and mannitol), proteins, adjuvants, polypeptides or amino acids such as glycine, antioxidants, chelating agents such as EDTA or glutathione, and/or preservatives.

Further, one or more other active ingredients may, but need not, be included in the pharmaceutical compositions provided herein. For instance, the compounds of the invention may advantageously be employed in combination with an antibiotic, antifungal, or antiviral agent, antihistamine, a non-steroidal anti-inflammatory drug, a disease modifying antirheumatic drug, a cytostatic drug, a drug with smooth muscle modulatory activity, an inhibitor of one or more of the enzymes that process the compounds of the present invention and lead to a decrease in their efficacy (for example, a cEH inhibitor), or mixtures of these.

Pharmaceutical compositions may be formulated for any appropriate route of administration including, for example, topical (for example, transdermal or ocular), oral, buccal, nasal, vaginal, rectal or parenteral administration. The term parenteral as used herein includes subcutaneous, intradermal, intravascular (for example, intravenous), intramuscular, spinal, intracranial, intrathecal, intraocular, periocular, intraorbital, intrasynovial and intraperitoneal injection, as well as any similar injection or infusion technique. In certain embodiments, compositions in a form suitable for oral use or parenteral use are preferred. Suitable oral forms include, for example, tablets, troches, lozenges, aqueous or oily suspensions, dispersible powders or granules, emulsions, hard or soft capsules, or syrups or elixirs. Within yet other embodiments, compositions provided herein may be formulated as a lyophilizate. Formulation for topical administration may be preferred for certain conditions such as in the treatment of skin conditions (for example, burns, itches or skin cancers).

Particularly preferred formulations for parenteral administration are liposomal formulations of the active compound (i.e. where the active compounds are contained or encapsulated in liposomes).

Liposomes comprising the compounds of the invention can be made by standard techniques including forming an organic solution having one or more compounds of the invention dissolved therein, contacting the organic solution with an aqueous solution and providing conditions for formation of a liposome therefrom.

A liposome may have a pH sensitivity of about pH 7.0, which means that the liposome is unstable below pH 7.0 such that the lipid bilayer of the liposome is disrupted below pH 7.0. A liposome may have a diameter ranging between about 50 nm and 200 μm. Accordingly, the liposome may be a small, sonicated unilamellar vesicle (SUV), a large unilamellar vesicle (LUV), or a liposome prepared by reverse phase evaporation (a REV), by french press (a FPV) or by ether injection (an EIV). Methods of preparing liposomes of such sizes, including methods of fractionating and purifying liposomes of the desired size, are known to a person skilled in the art.

A liposome may be unilamellar with respect to the liposome lipid bilayer. However, it will be understood that the liposome may comprise more than one lipid bilayer. Therefore, in one embodiment, the liposome may be a multilamellar vesicle such as a large, vortexed multilamellar vesicle (MLV).

A compound for providing the liposome with a charge for binding the liposome to a target cell may be advantageous for improving the fusion between the target lipid bilayer and the liposome bilayer. For example, DOTAP is particularly useful as a binding means for binding the liposome lipid bilayer to a target cell.

In one embodiment, a compound of the invention may be comprised in a layer of the lipid bilayer of the liposome. In this embodiment, a less hydrophobic portion of the molecule may be in contact with an inner aqueous core of the liposome, or in contact with an aqueous solution in which the liposome is contained.

Where the compound of the invention is provided in the form of a liposome as discussed above, in one embodiment the compound is administered to an individual requiring treatment by administration of a liposome including the compound, or a composition including said liposome, to an individual by injection.

Compositions intended for oral use may further comprise one or more components such as sweetening agents, flavouring agents, colouring agents and/or preserving agents in order to provide appealing and palatable preparations. Tablets contain the active ingredient in admixture with physiologically acceptable excipients that are suitable for the manufacture of tablets. Such excipients include, for example, inert diluents such as calcium carbonate, sodium carbonate, lactose, calcium phosphate or sodium phosphate, granulating and disintegrating agents such as corn starch or alginic acid, binding agents such as starch, gelatin or acacia, and lubricating agents such as magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules wherein the active ingredient is mixed with an inert solid diluent such as calcium carbonate, calcium phosphate or kaolin, or as soft gelatin capsules wherein the active ingredient is mixed with water or an oil medium such as peanut oil, liquid paraffin or olive oil.

Aqueous suspensions contain the active ingredient(s) in admixture with excipients suitable for the manufacture of aqueous suspensions. Such excipients include suspending agents such as sodium carboxymethylcellulose, methylcellulose, hydroxypropylmethylcellulose, sodium alginate, polyvinylpyrrolidone, gum tragacanth and gum acacia, and dispersing or wetting agents such as naturally-occurring phosphatides (for example, lecithin), condensation products of an alkylene oxide with fatty acids such as polyoxyethylene stearate, condensation products of ethylene oxide with long chain aliphatic alcohols such as heptadecaethyleneoxycetanol, condensation products of ethylene oxide with partial esters derived from fatty acids and a hexitol such as polyoxyethylene sorbitol mono-oleate, or condensation products of ethylene oxide with partial esters derived from fatty acids and hexitol anhydrides such as polyethylene sorbitan monooleate. Aqueous suspensions may also comprise one or more preservatives, for example ethyl, or n-propyl p-hydroxybenzoate, one or more colouring agents, one or more flavouring agents, and one or more sweetening agents, such as sucrose or saccharin.

Oily suspensions may be formulated by suspending the active ingredients in a vegetable oil such as arachis oil, olive oil, sesame oil or coconut oil, or in a mineral oil such as liquid paraffin. The oily suspensions may contain a thickening agent such as beeswax, hard paraffin or cetyl alcohol. Sweetening agents such as those set forth above, and/or flavoring agents may be added to provide palatable oral preparations. Such suspensions may be preserved by the addition of an antioxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, such as sweetening, flavouring and colouring agents, may also be present.

Pharmaceutical compositions may also be in the form of oil-in-water emulsions. The oily phase may be a vegetable oil such as olive oil or arachis oil, a mineral oil such as liquid paraffin, or a mixture thereof. Suitable emulsifying agents include naturally-occurring gums such as gum acacia or gum tragacanth, naturally-occurring phosphatides such as soy bean lecithin, and esters or partial esters derived from fatty acids and hexitol, anhydrides such as sorbitan monoleate, and condensation products of partial esters derived from fatty acids and hexitol with ethylene oxide such as polyoxyethylene sorbitan monoleate. An emulsion may also comprise one or more sweetening and/or flavoring agents.

Syrups and elixirs may be formulated with sweetening agents, such as glycerol, propylene glycol, sorbitol or sucrose. Such formulations may also comprise one or more demulcents, preservatives, flavouring agents and/or colouring agents.

Compounds may be formulated for local or topical administration, such as for topical application to the skin or mucous membranes, such as in the eye. Formulations for topical administration typically comprise a topical vehicle combined with active agent(s), with or without additional optional components. Suitable topical vehicles and additional components are well known in the art, and it will be apparent that the choice of a vehicle will depend on the particular physical form and mode of delivery. Topical vehicles include organic solvents such as alcohols (for example, ethanol, iso-propyl alcohol or glycerin), glycols such as butylene, isoprene or propylene glycol, aliphatic alcohols such as lanolin, mixtures of water and organic solvents and mixtures of organic solvents such as alcohol and glycerin, lipid-based materials such as fatty acids, acylglycerols including oils such as mineral oil, and fats of natural or synthetic origin, phosphoglycerides, sphingolipids and waxes, protein-based materials such as collagen and gelatine, silicone-based materials (both nonvolatile and volatile), and hydrocarbon-based materials such as microsponges and polymer matrices.

A composition may further include one or more components adapted to improve the stability or effectiveness of the applied formulation, such as stabilizing agents, suspending agents, emulsifying agents, viscosity adjusters, gelling agents, preservatives, antioxidants, skin penetration enhancers, moisturizers and sustained release materials. Examples of such components are described in Martindale—The Extra Pharmacopoeia (Pharmaceutical Press, London 1993) and Martin (ed.), Remington's Pharmaceutical Sciences. Formulations may comprise microcapsules, such as hydroxymethylcellulose or gelatin-microcapsules, liposomes, albumin microspheres, microemulsions, nanoparticles or nanocapsules.

A topical formulation may be prepared in a variety of physical forms including, for example, solids, pastes, creams, foams, lotions, gels, powders, aqueous liquids, emulsions, sprays and skin patches. The physical appearance and viscosity of such forms can be governed by the presence and amount of emulsifier(s) and viscosity adjuster(s) present in the formulation. Solids are generally firm and non-pourable and commonly are formulated as bars or sticks, or in particulate form. Solids can be opaque or transparent, and optionally can contain solvents, emulsifiers, moisturizers, emollients, fragrances, dyes/colorants, preservatives and other active ingredients that increase or enhance the efficacy of the final product. Creams and lotions are often similar to one another, differing mainly in their viscosity. Both lotions and creams may be opaque, translucent or clear and often contain emulsifiers, solvents, and viscosity adjusting agents, as well as moisturizers, emollients, fragrances, dyes/colorants, preservatives and other active ingredients that increase or enhance the efficacy of the final product. Gels can be prepared with a range of viscosities, from thick or high viscosity to thin or low viscosity. These formulations, like those of lotions and creams, may also contain solvents, emulsifiers, moisturizers, emollients, fragrances, dyes/colorants, preservatives and other active ingredients that increase or enhance the efficacy of the final product. Liquids are thinner than creams, lotions, or gels, and often do not contain emulsifiers. Liquid topical products often contain solvents, emulsifiers, moisturizers, emollients, fragrances, dyes/colorants, preservatives and other active ingredients that increase or enhance the efficacy of the final product.

Emulsifiers for use in topical formulations include, but are not limited to, ionic emulsifiers, cetearyl alcohol, non-ionic emulsifiers like polyoxyethylene oleyl ether, PEG-40 stearate, ceteareth-12, ceteareth-20, ceteareth-30, ceteareth alcohol, PEG-100 stearate and glyceryl stearate. Suitable viscosity adjusting agents include, but are not limited to, protective colloids or nonionic gums such as hydroxyethylcellulose, xanthan gum, magnesium aluminum silicate, silica, microcrystalline wax, beeswax, paraffin, and cetyl palmitate. A gel composition may be formed by the addition of a gelling agent such as chitosan, methyl cellulose, ethyl cellulose, polyvinyl alcohol, polyquaterniums, hydroxyethylcellulose, hydroxypropylcellulose, hydroxypropylmethylcellulose, carbomer or ammoniated glycyrrhizinate. Suitable surfactants include, but are not limited to, nonionic, amphoteric, ionic and anionic surfactants. For example, one or more of dimethicone copolyol, polysorbate 20, polysorbate 40, polysorbate 60, polysorbate 80, lauramide DEA, cocamide DEA, and cocamide MEA, oleyl betaine, cocamidopropyl phosphatidyl PG-dimonium chloride, and ammonium laureth sulfate may be used within topical formulations.

Preservatives include, but are not limited to, antimicrobials such as methylparaben, propylparaben, sorbic acid, benzoic acid, and formaldehyde, as well as physical stabilizers and antioxidants such as vitamin E, sodium ascorbate/ascorbic acid and propyl gallate. Suitable moisturizers include, but are not limited to, lactic acid and other hydroxy acids and their salts, glycerin, propylene glycol, and butylene glycol. Suitable emollients include lanolin alcohol, lanolin, lanolin derivatives, cholesterol, petrolatum, isostearyl neopentanoate and mineral oils. Suitable fragrances and colours include, but are not limited to, FD&C Red No. 40 and FD&C Yellow No. 5. Other suitable additional ingredients that may be included in a topical formulation include, but are not limited to, abrasives, absorbents, anticaking agents, antifoaming agents, antistatic agents, astringents (such as witch hazel), alcohol and herbal extracts such as chamomile extract, binders/excipients, buffering agents, chelating agents, film forming agents, conditioning agents, propellants, opacifying agents, pH adjusters and protectants.

Typical modes of delivery for topical compositions include application using the fingers, application using a physical applicator such as a cloth, tissue, swab, stick or brush, spraying including mist, aerosol or foam spraying, dropper application, sprinkling, soaking, and rinsing. Controlled release vehicles can also be used, and compositions may be formulated for transdermal administration (for example, as a transdermal patch).

A pharmaceutical composition may be formulated as inhaled formulations, including sprays, mists, or aerosols. For inhalation formulations, the compounds provided herein may be delivered via any inhalation methods known to a person skilled in the art. Such inhalation methods and devices include, but are not limited to, metered dose inhalers with propellants such as CFC or HFA or propellants that are physiologically and environmentally acceptable. Other suitable devices are breath operated inhalers, multidose dry powder inhalers and aerosol nebulizers. Aerosol formulations for use in the subject method typically include propellants, surfactants and co-solvents and may be filled into conventional aerosol containers that are closed by a suitable metering valve.

Inhalant compositions may comprise liquid or powdered compositions containing the active ingredient that are suitable for nebulization and intrabronchial use, or aerosol compositions administered via an aerosol unit dispensing metered doses. Suitable liquid compositions comprise the active ingredient in an aqueous, pharmaceutically acceptable inhalant solvent such as isotonic saline or bacteriostatic water. The solutions are administered by means of a pump or squeeze-actuated nebulized spray dispenser, or by any other conventional means for causing or enabling the requisite dosage amount of the liquid composition to be inhaled into the patient's lungs. Suitable formulations, wherein the carrier is a liquid, for administration, as for example, a nasal spray or as nasal drops, include aqueous or oily solutions of the active ingredient.

Pharmaceutical compositions may also be prepared in the form of suppositories such as for rectal administration. Such compositions can be prepared by mixing the drug with a suitable non-irritating excipient that is solid at ordinary temperatures but liquid at the rectal temperature and will therefore melt in the rectum to release the drug. Suitable excipients include, for example, cocoa butter and polyethylene glycols.

Pharmaceutical compositions may be formulated as sustained release formulations such as a capsule that creates a slow release of modulator following administration. Such formulations may generally be prepared using well-known technology and administered by, for example, oral, rectal or subcutaneous implantation, or by implantation at the desired target site. Carriers for use within such formulations are biocompatible, and may also be biodegradable. Preferably, the formulation provides a relatively constant level of modulator release. The amount of modulator contained within a sustained release formulation depends upon, for example, the site of implantation, the rate and expected duration of release and the nature of the condition to be treated or prevented.

For the treatment of proliferative disorders, especially metastatic disorders, the dose of the biologically active compound according to the invention may vary within wide limits and may be adjusted to individual requirements. Active compounds according to the present invention are generally administered in a therapeutically effective amount. Preferred doses range from about 0.1 mg to about 140 mg per kilogram of body weight per day (e.g. about 0.5 mg to about 7 g per patient per day). The daily dose may be administered as a single dose or in a plurality of doses. The amount of active ingredient that may be combined with the carrier materials to produce a single dosage form will vary depending upon the host treated and the particular mode of administration. Dosage unit forms will generally contain between about 1 mg to about 500 mg of an active ingredient.

It will be understood, however, that the specific dose level for any particular patient will depend upon a variety of factors including the activity of the specific compound employed, the age, body weight, general health, sex, diet, time of administration, route of administration, and rate of excretion, drug combination (i.e. other drugs being used to treat the patient), and the severity of the particular disorder undergoing therapy.

The terms “therapeutically effective amount” or “effective amount” refer to an amount of the compound of formula (I) that results in an improvement or remediation of the symptoms of a proliferative and/or metastatic disorder.

Preferred compounds of the invention will have certain pharmacological properties. Such properties include, but are not limited to oral bioavailability, such that the preferred oral dosage forms discussed above can provide therapeutically effective levels of the compound in vivo.

The compounds of the present invention are preferably administered to a patient (for example, a human) orally or parenterally, and are present within at least one body fluid or tissue of the patient. Accordingly, the present invention further provides methods for treating patients suffering from proliferative disorders (including metastatic disorders). As used herein, the term “treatment” encompasses both disorder-modifying treatment and symptomatic treatment. It refers to therapeutic treatment, i.e. after the onset of symptoms, in order to reduce the severity and/or duration of symptoms, and/or to cure the condition or disorder. As used herein, the term “prevention” encompasses prophylactic treatment, i.e. before the onset of symptoms, in order to prevent, delay or reduce the severity of symptoms and/or the condition or disorder. Patients may include but are not limited to primates, especially humans, domesticated companion animals such as dogs, cats, horses, and livestock such as cattle, pigs, sheep, with dosages as described herein.

Compounds of the present invention may be useful for the treatment and/or prevention of conditions and disorders associated with cell proliferation (including metastasis). Accordingly, the present invention also relates to a method of treating or preventing a proliferative disorder in a patient including administration to the patient of a therapeutically effective amount of a compound of formula (I), or a pharmaceutically-acceptable salt, solvate or hydrate thereof. The present invention also relates to the use of a therapeutically effective amount of a compound of formula (I), or a pharmaceutically-acceptable salt, solvate or hydrate thereof, for treating or preventing a proliferative disorder. The present invention also provides a pharmaceutical composition for use in treating or preventing a proliferative disorder, in any of the embodiments described in the specification. The present invention also relates to the use of a therapeutically effective amount of a compound of formula (I), or a pharmaceutically acceptable salt, solvate or hydrate thereof, for the manufacture of a medicament for treating or preventing a proliferative disorder.

The present invention also relates to a compound of formula (I), or a pharmaceutically acceptable salt, solvate or hydrate thereof, when used in a method of treating or preventing a proliferative disorder. The present invention also relates to a composition having an active ingredient for use in treating or preventing a proliferative disorder, wherein the active ingredient is a compound of formula (I), or a pharmaceutically acceptable salt, solvate or hydrate thereof. The present invention also relates to the use of a pharmaceutical composition containing a compound of the formula (I), or a pharmaceutically acceptable salt, solvate or hydrate thereof, in treating or preventing a proliferative disorder, such as described above. In one embodiment, the compound of formula (I) is essentially the only active ingredient of the composition. In one embodiment, the proliferative disorder is a metastatic disorder.

Examples of conditions and disorders associated with cell proliferation include tumours or neoplasms, where proliferation of cells is uncontrolled and progressive. Some such uncontrolled proliferating cells are benign, but others are termed “malignant” and may lead to death of the organism. Malignant neoplasms or “cancers” are distinguished from benign growths in that, in addition to exhibiting aggressive cellular proliferation, they may invade surrounding tissues and metastasize. Moreover, malignant neoplasms are characterized in that they show a greater loss of differentiation (greater “dedifferentiation”), and greater loss of their organization relative to one another and their surrounding tissues. This property is also called “anaplasia”. Neoplasms treatable by the present invention also include solid phase tumours/malignancies, i. e. carcinomas, locally advanced tumours and human soft tissue sarcomas. Carcinomas include those malignant neoplasms derived from epithelial cells that infiltrate (invade) the surrounding tissues and give rise to metastastic cancers, including lymphatic metastases. The compounds of the present invention have been found to be particularly effective in decreasing cancer development and against metastatic cancers (including in models of cell proliferation and migration). The compounds of the present invention have also been found to be particularly effective at killing primary cancer cells.

Adenocarcinomas are carcinomas derived from glandular tissue, or which form recognizable glandular structures. Another broad category of cancers includes sarcomas, which are tumours whose cells are embedded in a fibrillar or homogeneous substance like embryonic connective tissue.

The invention also enables treatment of cancers of the myeloid or lymphoid systems, including leukemias, lymphomas and other cancers that typically do not present as a tumour mass, but are distributed in the vascular or lymphoreticular systems.

The type of cancer or tumour cells that may be amenable to treatment according to the invention include, for example, breast, colon, lung, and prostate cancers, gastrointestinal cancers including esophageal cancer, stomach cancer, colorectal cancer, polyps associated with colorectal neoplasms, pancreatic cancer and gallbladder cancer, cancer of the adrenal cortex, ACTH-producing tumour, bladder cancer, brain cancer including intrinsic brain tumours, neuroblastomas, astrocytic brain tumours, gliomas, and metastatic tumour cell invasion of the central nervous system, Ewing's sarcoma, head and neck cancer including mouth cancer and larynx cancer, kidney cancer including renal cell carcinoma, liver cancer, lung cancer including small and non-small cell lung cancers, malignant peritoneal effusion, malignant pleural effusion, skin cancers including malignant melanoma, tumour progression of human skin keratinocytes, squamous cell carcinoma, basal cell carcinoma, and hemangiopericytoma, mesothelioma, Kaposi's sarcoma, bone cancer including osteomas and sarcomas such as fibrosarcoma and osteosarcoma, cancers of the female reproductive tract including uterine cancer, endometrial cancer, ovarian cancer, ovarian (germ cell) cancer and solid tumours in the ovarian follicle, vaginal cancer, cancer of the vulva, and cervical cancer, breast cancer (small cell and ductal), penile cancer, retinoblastoma, testicular cancer, thyroid cancer, trophoblastic neoplasms, and Wilms' tumour.

The present invention also relates to a method of inducing apoptosis in a cell, especially a cell undergoing cell division, the method including contacting the cell with a compound of formula (I) as discussed herein, or a mixture thereof, or a pharmaceutical composition as discussed herein.

The present invention also relates to a method of inhibiting cell migration, the method including contacting the cell with a compound of formula (I) as discussed herein, or a mixture thereof, or a pharmaceutical composition as discussed herein.

It is also within the present invention that the compounds according to the invention are used as or for the manufacture of a diagnostic agent, whereby such diagnostic agent is for the diagnosis of the disorders and conditions which can be addressed by the compounds of the present invention for therapeutic purposes as disclosed herein.

For various applications, the compounds of the invention can be labelled by isotopes, fluorescence or luminescence markers, antibodies or antibody fragments, any other affinity label like nanobodies, aptamers, peptides etc., enzymes or enzyme substrates. These labelled compounds of this invention are useful for mapping the location of receptors in vivo, ex vivo, in vitro and in situ such as in tissue sections via autoradiography and as radiotracers for positron emission tomography (PET) imaging, single photon emission computerized tomography (SPECT) and the like, to characterize those receptors in living subjects or other materials. The labelled compounds according to the present invention may be used in therapy, diagnosis and other applications such as research tools in vivo and in vitro, in particular the applications disclosed herein.

### EXAMPLES

### Synthesis

### tert-Butyl-(3-bromopropyl)carbamate (2)

3-bromopropylamine hydrobromide 1 (4.000 g, 18.3 mmol) and di-tert-butyl dicarbonate (7.987 g, 36.6 mmol) were dissolved in anhydrous DCM (100 mL) under nitrogen gas flow. To the stirred solution was added DIPEA (3.50 mL, 20.2 mmol). After 12 hours the solvent was removed and the residue dissolved in ethanol (10 mL) to which imidazole (18.7 mmol) was added and stirred for 30 minutes. The mixture was diluted with chloroform (100 mL) and washed with 1% HCl solution (3×50 mL). The organic phase was dried with sodium sulphate and evaporated to yield 2 (4.389 g, 95%). ¹H NMR (500 MHz, CDCl₃): δ 3.44 (t, J=6.5 Hz, 2H), 3.27 (t, J=6.5 Hz, 2H), 2.05 (p, J=6.5 Hz, 2H), 1.44 (s, 9H).

### S-{3-[(tert-Butoxycarbonyl)amino]propyl} ethanethioate (3)

Potassium thioacetate (2.207 g, 19.3 mmol) was added to a stirred solution of 2 (4.184 g, 17.6 mmol) in DMF (50 mL) and stirred for 12 hours at RT. Dichloromethane (150 mL) was added and washed with water (3×300 mL). The combined fractions were dried with sodium sulphate and evaporated, yielding crude 3 as a solid (3.630 g). ¹H NMR (500 MHz, CDCl₃): δ 4.78 (br, 1H), 3.15 (t, J=67.0 Hz, 2H), 2.89 (q, J=7.0 Hz, 2H), 2.33 (s, 3H), 1.75 (p, J=7.0 Hz, 2H), 1.44 (s, 9H).

### tert-Butyl-(3-sulfanylpropyl)carbamate (4)

Potassium carbonate (4.312 g, 31.2 mmoL) was added to a stirred solution of 3 (3.630 g, 15.6 mmoL) in 50:50 water:methanol (80 mL) under nitrogen gas flow. After an hour, the mixture was poured into ethyl acetate (100 mL) and washed with water (3×100 mL). The organic phase was dried over sodium sulfate and evaporated and resultant crude purified on silica gel by stepwise gradient elution with dichloromethane/ethyl acetate, yielding 4 (2.431 g, 82%). ¹H NMR (500 MHz, CDCl₃): δ 4.58 (br, 1H), 3.24 (q, J=6.5 Hz, 2H), 2.56 (q, J=7.5 Hz, 2H), 1.79 (p, J=7.0 Hz, 2H), 1.44 (s, 9H).

### Ethyl 12-bromododecanoate (6)

Acetyl chloride (3.84 ml, 53.7 mmol) added dropwise to a stirred solution of 12-bromododecanoic acid 5 (5.000 g, 17.9 mmol). The mixture was stirred for 3 hours at room temperature. The reaction was evaporated in vacuo and the residue dissolved into diethyl ether (300 mL) and washed with 0.75M NaOH (3×300 mL). The organic phase was dried over sodium sulfate and evaporated to yield 8 as an oil (6.111 g, 90%). ¹H NMR (500 MHz, CDCl₃): δ 4.22 (q, J=7.0 Hz, 2H), 3.41 (t, J=7.0 Hz, 2H), 2.89 (t, J=7.5 Hz, 2H), 1.86 (p, J=7.5 Hz, 2H), 1.62 (p, J=7.5 Hz, 2H), 1.42 (p, J=7.5 Hz, 2H), 1.35-1.20 (m, 15H).

### Ethyl 12-({3-[(tert-butoxycarbonyl)amino]propyl}sulfanyl)dodecanoate (7)

6 (0.400 g, 1.3 mmol) and 4 (0.249 g, 1.3 mmol) were dissolved in dry DMF (10 mL) under nitrogen gas. The solution was bubbled with dry nitrogen gas and potassium carbonate (0.540 g, 3.9 mmol) was added. After 12 hours water (30 mL) was added and compound extracted into diethyl ether (3×30 mL). The combined ether fractions were washed with water, brine, and then evaporated. The crude product was purified on silica gel by stepwise gradient elution with dichloromethane/ethyl acetate, yielding 9 (0.275 g, 50%). ¹H NMR (500 MHz, CDCl₃): δ 4.63 (br, 1H), 4.13 (q, J=7.0 Hz, 2H), 3.20 (q, J=7.0 Hz, 2H), 2.53 (t, J=7.0 Hz, 2H), 2.49 (t, J=7.0 Hz, 2H), 2.28 (t, J=7.0 Hz, 2H), 1.76 (p, J=7.0 Hz, 2H), 1.61 (p, J=7.5 Hz, 2H), 1.57 (p, J=7.5 Hz, 2H), 1.44 (s, 9H), 1.40-1.20 (m, 17H).

### General Procedure for the Removal of the N-Boc Group

The BOC protected amine (0.59 mmol) was dissolved in dichloromethane (16 mL) at 0° C. TFA (4 mL) was added drop wise achieving a final concentration of approximately 2.5 M TFA. The reaction was allowed to warm to RT. After 1 hour the solvent was removed and saturated sodium carbonate (50 mL) solution added. The product was extracted with dichloromethane (3×50 mL), and the combined fractions were washed with sat. aqueous sodium carbonate and brine, and then dried with sodium sulfate and evaporated to yield the amine.

### Ethyl 12-[(3-aminopropyl)sulfanyl]dodecanoate (8)

8 was prepared from 7 according to the general procedure for removal of the Boc group. Pale yellow solid, 86%. ¹H NMR (500 MHz, CDCl₃): δ 4.12 (q, J=7.0 Hz, 2H), 2.86 (t, J=6.5 Hz, 2H), 2.58 (t, J=7.0 Hz, 2H), 2.51 (t, J=7.0 Hz, 2H), 2.28 (t, J=7.0 Hz, 2H), 1.77 (p, J=7.0 Hz, 2H), 1.65-1.50 (m, 4H), 1.40-1.20 (m, 17H).

### General Procedure for the Formation of Urea Groups.

Appropriately substituted aryl isocyanate (0.32 mmol) was added to a stirred solution of the amine (0.32 mmol) in dry THF (5 mL) under nitrogen for 3 hours. The solvent was removed in vacuo and the crude product was purified on silica gel by stepwise gradient elution with dichloromethane/ethyl acetate.

### Ethyl 12-{[3-({[4-chloro-3-(trifluoromethyl)phenyl]carbamoyl}amino)propyl]sulfanyl}dodecanoate (9)

9 was prepared from 8 according to the general procedure for the formation of the urea group.

White solid, 56%. ¹H NMR (500 MHz, CDCl₃): δ 7.66 (d, J=2.5 Hz, 1H), 7.58 (dd, J=8.5, 2.5 Hz, 1H), 7.38 (d, J=8.5 Hz, 1H), 6.76 (br, 1H), 4.98 (t, J=5.5 Hz, 1H), 4.12 (q, J=7.0 Hz, 2H), 3.38 (q, J=7.0 Hz, 2H), 2.59 (t, J=7.0 Hz, 2H), 2.50 (t, J=7.0 Hz, 2H), 2.30 (t, J=7.5 Hz, 2H), 1.84 (p, J=7.0 Hz, 2H), 1.65-1.50 (m, 4H), 1.40-1.20 (m, 17H).

### Ethyl 12-[(3-{[(4-methylphenyl)carbamoyl]amino}propyl)sulfanyl]dodecanoate (10)

10 was prepared from 8 according to the general procedure for the formation of the urea group.

White solid, 48%. ¹H NMR (500 MHz, CDCl₃): δ 7.17-7.14 (m, 2H), 7.12-7.09 (m, 2H), 6.53 (br, 1H), 5.07 (t, J=6.0 Hz, 1H), 4.12 (q, J=7.0 Hz, 2H), 3.32 (q, J=7.0 Hz, 2H), 2.53 (t, J=7.0 Hz, 2H), 2.46 (t, J=7.0 Hz, 2H), 2.30-2.25 (m, 5H), 1.78 (p, J=7.0 Hz, 2H), 1.61 (p, J=7.0 Hz, 2H), 1.53 (p, J=6.5 Hz, 2H), 1.40-1.20 (m, 17H).

### General Procedure for the Hydrolysis of the Ester Groups.

The ester (0.18 mmol) was dissolved in ethanol (5 mL) and 1 M NaOH (3 mL) was added. The reaction was heated to 40° C. and stirred for 3 hours. The reaction volume was reduced to half in vacuo, then cooled and acidified with 1 M HCl solution to pH 1. The precipitate was extracted into dichloromethane (3×25 mL), washed with brine, dried over sodium sulphate and evaporated to yield the carboxylic acids.

### 12-{[3-({[4-Chloro-3-(trifluoromethyl)phenyl]carbamoyl}amino)propyl]sulfanyl}dodecanoic acid (CP01)

CP01 was prepared from 9 according to the general procedure for the hydrolysis of the ester group.

Waxy solid, 80%. ¹H NMR (500 MHz, CDCl₃): δ 7.65 (d, J=2.5 Hz, 1H), 7.55 (d, J=8.5 Hz, 1H), 7.39 (d, J=8.5 Hz, 1H), 3.38 (t, J=7.5 Hz, 2H), 2.560 (br, 2H), 2.50 (br, 2H), 2.36 (t, J=7.5 Hz, 2H), 1.84 (p, J=6.5 Hz, 2H), 1.64 (p, J=7.0 Hz, 2H), 1.56 (p, J=7.0 Hz, 2H), 1.40-1.20 (m, 14H).

### 12-[(3-{[(4-Methylphenyl)carbamoyl]amino}propyl)sulfanyl]dodecanoic acid (CP02)

CP02 was prepared from 10 according to the general procedure for the hydrolysis of the ester group.

White solid, 85%. ¹H NMR (500 MHz, DMSO-d₆): δ 8.38 (br, 1H), 7.26-7.23 (m, 2H), 7.00-6.97 (m, 2H), 6.23 (br, 1H), 3.12 (q, J=6.5 Hz, 2H), 2.55-2.40 (m, 4H), 2.19 (s, 3H), 2.15 (t, J=7.5 Hz, 2H), 1.64 (p, J=7.0 Hz, 2H), 1.50-1.40 (m, 4H), 1.31 (p, J=6.5 Hz, 2H), 1.35-1.20 (m, 12H).

### 2-(13-Hydroxytridecyl)-1H-isoindole-1,3(2H)-dione (12)

13-bromotridecan-1-ol 11 (4.854 g, 17.4 mmol) and potassium phthalate (4.311 g, 23.0 mmol) were dissolved in anhydrous DMF (50 mL) under nitrogen gas. The reaction was stirred at 70° C. for 12 hours. The mixture was diluted with water and extracted into ethyl acetate (3×200 mL). The combined extracts were washed with brine and dried over sodium sulfate. The solvent was removed in vacuo to yield 6.059 g of a crude product. ¹H NMR (500 MHz, CDCl₃): δ 7.85-7.83 (m, 2H), 7.72-7.70 (m, 2H), 3.75-7.60 (m, 4H), 1.67 (p, J=7.0 Hz, 2H), 1.57 (p, J=6.5 Hz, 2H), 1.40-1.20 (m, 18H).

### 13-Aminotridecan-1-ol (13)

A solution of 12 (6.053 g, 17.5 mmol) and hydrazine monohydrate (2.55 mL, 52.5 mmol) in ethanol (200 mL) were refluxed overnight, after which the solvent was removed in vacuo. The residue was dissolved in dichloromethane (200 mL) and washed with 1 M NaOH (200 mL) and brine, and evaporated to dryness yielding 13 (3.402 g) as a crude product. ¹H NMR (500 MHz, CDCl₃): δ 3.64 (t, J=7.0 Hz, 2H), 2.68 (t, J=7.0 Hz, 2H), 1.57 (p, J=7.0 Hz, 2H), 1.43 (t, J=7.0 Hz, 2H), 1.40-1.35 (m, 18H).

### 13-Bromotridecan-1-amine hydrobromide (14)

13 (3.402 g, 15.8 mmol) was refluxed in 50% hydrobromic acid solution (100 mL) for 24 hours, after which the reaction was cooled at room temperature and the resulting precipitate filtered. The precipitate was recrystallised in acetone and washed with cold diethyl ether, yielding 14 as a dry brown powder (4.056 g, 72%). ¹H NMR (500 MHz, DMSO-d₆): δ 3.51 (t, J=7.0 Hz, 2H), 2.75 (br, 2H), 1.77 (t, J=7.0 Hz, 2H), 1.50 (br, 2H), 1.36 (t, J=7.0 Hz, 2H), 1.35-1.20 (m, 16H).

### tert-Butyl (13-bromotridecyl)carbamate (15)

14 (0.502 g, 1.39 mmol) and di-tert-butyl dicarbonate (0.456 g, 2.09 mmol) were dissolved in anhydrous dichloromethane (10 mL) under nitrogen gas. DIPEA was added (270 μL, 1.53 mmol) and effervescence was observed. The reaction was stirred overnight, after which imidazole (0.057 g, 0.84 mmol) was added and left to react for 30 minutes. The reaction mixture was diluted with dichloromethane (50 mL), washed with 1% HCl solution (3×50 mL), dried over sodium sulfate and evaporated to dryness, yielding 15 as a yellow, viscous oil (0.455 g, 86%). ¹H NMR (500 MHz, CDCl₃): δ 4.48 (br, 1H), 3.41 (t, J=7.0 Hz, 2H), 3.10 (t, J=7.0 Hz, 2H), 1.85 (p, J=7.0 Hz, 2H), 1.50-1.40 (m, 13H), 1.35-1.25 (m, 16H).

### Methyl ({13-[(tert-butoxycarbonyl)amino]tridecyl}sulfanyl)acetate (16)

(0.205 g, 0.54 mmol) and methyl thioglycolate (47.3 μL, 0.54 mmol) were dissolved in dry DMF (5 mL) under nitrogen gas. Potassium carbonate (0.220 g, 1.59 mmol) was added the and reaction mixture was stirred overnight. Ethyl actetate (100 mL) was added and washed with water (3×300 mL). The organic layer was dried over sodium carbonate and solvent removed in vacuo. The crude product was purified on silica gel by stepwise gradient elution with dichloromethane/ethyl acetate, yielding 16 as a pale yellow oil (0.174 g, 82%). ¹H NMR (500 MHz, CDCl₃): δ 4.49 (br, 1H), 3.74 (s, 3H), 3.22 (m, 2H), 3.10 (q, J=6.5 Hz, 2H), 2.62 (t, J=7.5 Hz, 2H), 1.59 (p, J=7.5 Hz, 2H), 1.50-1.40 (m, 11H), 1.36 (p, J=7.5 Hz, 2H), 1.40-1.30 (m, 16H).

### Methyl [(13-aminotridecyl)sulfanyl]acetate (17)

17 was prepared from 16 according to the general procedure for removal of the Boc group.

Oily residue obtained, yield 84%. ¹H NMR (500 MHz, CDCl₃): δ 3.74 (s, 3H), 3.22 (m, 2H), 2.68 (t, J=7.0 Hz, 2H), 2.62 (t, J=7.0 Hz, 2H), 1.59 (p, J=7.5 Hz, 2H), 1.43 (p, J=7.0 Hz, 2H), 1.37 (p, J=7.5 Hz, 2H), 1.30-1.20 (m, 16H).

### Methyl {[13-({[4-chloro-3-(trifluoromethyl)phenyl]carbamoyl}amino)tridecyl]sulfanyl}acetate (18)

18 was prepared from 17 by the general procedure for the formation of ureas.

Waxy solid, yield 72%. ¹H NMR (500 MHz, CDCl₃): δ 7.68 (d, J=2.5 Hz, 1H), 7.57 (dd, J=8.5, 2.5 Hz, 1H), 7.40 (d, J=8.5 Hz, 1H), 6.42 (br, 1H), 4.65 (br, 1H), 3.75 (s, 3H), 3.29-3.24 (m, 4H), 2.63 (t, J=7.0 Hz, 2H), 1.62-1.50 (m, 4H), 1.40-1.20 (m, 18H)

### Methyl [(5-{[(4-methylphenyl)carbamoyl]amino}tridecyl)sulfanyl]acetate (19)

19 was prepared from 17 by the general procedure for the formation of ureas.

Waxy solid, yield 86%. ¹H NMR (500 MHz, CDCl₃): δ 7.14 (m, 4H), 5.99 (br, 1H), 4.58 (br, 1H), 3.74 (s, 3H), 3.25-3.20 (s, 4H), 2.63 (t, J=7.5 Hz, 2H), 2.32 (s, 3H), 1.59 (p, J=7.5 Hz, 2H), 1.48 (p, J=7.5 Hz, 2H), 1.37 (p, J=7.5 Hz, 2H), 1.30-1.20 (m, 16H).

### {[13-({[4-Chloro-3-(trifluoromethyl)phenyl]carbamoyl}amino)tridecyl]sulfanyl}acetic acid (CP03)

CP03 was prepared from 18 by the general procedure given for the hydrolysis of esters. White solid, yield 93%. ¹H NMR (500 MHz, CDCl₃): δ 7.61 (d, J=2.5 Hz, 1H), 7.52 (dd, J=9.0, 2.5 Hz, 1H), 7.40 (d, J=9.0 Hz, 1H), 7.11 (br, 1H), 5.27 (br, 1H), 3.30-3.20 (m, 4H), 2.69 (t, J=7.5 Hz, 2H), 1.62 (p, J=7.5 Hz, 2H), 1.52 (p, J=7.0 Hz, 2H), 1.40-1.20 (m, 18H).

### [(5-{[(4-Methylphenyl)carbamoyl]amino}tridecyl)sulfanyl]acetic acid (CP04)

CP04 was prepared from 19 by the general procedure given for the hydrolysis of esters.

Waxy solid, yield 58%. ¹H NMR (500 MHz, CDCl₃): δ 7.59 (br, 1H), 7.14 (m, 2H), 7.09 (m, 2H), 5.04 (br, 1H), 3.25-3.20 (s, 4H), 2.67 (t, J=7.5 Hz, 2H), 2.33 (s, 3H), 1.64 (p, J=7.5 Hz, 2H), 1.55-1.45 (m, 2H), 1.41 (p, J=7.5 Hz, 2H), 1.30-1.20 (m, 16H).

### Methyl [(9-bromononyl)sulfanyl]acetate (21)

1,9-dibromononane (1421 μL, 6.99 mmol) and methyl thioglycolate (0.625 μL, 6.99 mmol) were dissolved in dry DMF (20 mL) under nitrogen gas. Potassium carbonate (2.899 g, 20.97 mmol) was added and reaction stirred overnight. Ethyl actetate (200 mL) was added and washed with water (3×300 mL), organic layer separated and dried over sodium sulfate and solvent removed in vacuo. The crude product was purified on silica gel by stepwise gradient elution with hexane/dichloromethane, yielding 21 as a pale yellow oil (0.9819 g, 45% yield). ¹H NMR (500 MHz, CDCl₃): δ 3.74 (s, 3H), 3.41 (t, J=7.0 Hz, 2H), 3.23 (s, 2H), 2.63 (t, J=7.5 Hz, 2H), 1.86 (p, J=7.0 Hz, 2H), 1.60 (p, J=7.0 Hz, 2H), 1.45-1.35 (m, 4H), 1.33-1.25 (m, 6H).

### Methyl 2,2-dimethyl-4-oxo-3-oxa-9,19-dithia-5-aza heneicosan-21-oate (22)

21 (0.9819 g, 3.154 mmol) and 4 (0.602 g, 3.154 mmol) were dissolved in dry DMF (20 mL) under nitrogen and potassium carbonate (1.308 g, 9.463 mmol) was added. The reaction was stirred overnight then diluted with ethyl acetate (100 mL) and washed with water (3×250 mL). The organic layer was dried over sodium sulfate and the solvent evapourated in vacuo. The crude product was purified on silica gel by stepwise gradient elution with dichloromethane/ethyl acetate, yielding 22 as an oil (0.754 g, 57%). ¹H NMR (500 MHz, CDCl₃): δ 4.63 (br, 1H), 3.74 (s, 3H), 3.25-3.15 (m, 4H), 2.62 (t, J=7.5 Hz, 2H), 2.53 (t, J=7.0 Hz, 2H), 2.49 (t, J=7.5 Hz, 2H), 1.77 (p, J=7.0 Hz, 2H), 1.60-1.50 (m, 4H), 1.44 (s, 9H), 1.40-1.30 (m, 4H), 1.30-1.25 (m, 6H).

### Methyl ({9-[(3-aminopropyl)sulfanyl]nonyl}sulfanyl)acetate (23)

23 was prepared from 22 according to the general procedure for removal of the Boc group. Oily residue obtained, yield 99%. ¹H NMR (500 MHz, CDCl₃): δ 3.74 (s, 3H), 3.22 (s, 2H), 2.80 (t, J=6.5 Hz, 2H), 2.62 (t, J=7.5 Hz, 2H), 2.56 (t, J=7.0 Hz, 2H), 2.51 (t, J=7.0 Hz, 2H), 1.73 (p, J=7.0 Hz, 2H), 1.60-1.50 (m, 4H), 1.40-1.30 (m, 4H), 1.30-1.25 (m, 6H).

### Methyl [(9-{[3-({[4-chloro-3-(trifluoromethyl)phenyl]carbamoyl}amino)propyl]sulfanyl}nonyl)sulfanyl]acetate (24)

24 was prepared from 23 according to the general procedure given for the formation of ureas.

Waxy solid, yield 78%. ¹H NMR (500 MHz, CDCl₃): (7.69 (d, J=2.5 Hz, 1H), 7.58 (dd, J=8.5, 2.5 Hz, 1H), 7.39 (d, J=8.5 Hz, 1H), 6.69 (br, 1H), 4.97 (br, 1H), 3.75 (s, 3H), 3.38 (q, J=6.5 Hz, 2H), 3.24 (s, 2H), 2.65-2.55 (m, 4H), 2.51 (t, J=7.0 Hz, 2H), 1.84 (p, J=7.0 Hz, 2H), 1.60-1.50 (m, 4H), 1.40-1.30 (m, 4H), 1.30-1.25 (m, 6H).

### Methyl ({9-[(3-{[(4-methylphenyl)carbamoyl]amino}propyl)sulfanyl]nonyl}sulfanyl) acetate (25)

25 was prepared from 23 according to the general procedure given for the formation of ureas.

Waxy solid, yield 99%. ¹H NMR (500 MHz, CDCl₃): δ 7.15 (m, 4H), 6.10 (br, 1H), 4.80 (br, 1H), 3.74 (s, 3H), 3.42 (q, J=6.5 Hz, 2H), 3.23 (s, 2H), 2.62 (t, J=7.5 Hz, 2H), 2.55 (t, J=7.0 Hz, 2H), 2.48 (t, J=7.5 Hz, 2H), 2.32 (s, 3H), 1.81 (p, J=7.0 Hz, 2H), 1.60-1.50 (m, 4H), 1.40-1.30 (m, 4H), 1.30-1.25 (m, 6H).

### [(9-{[3-({[4-Chloro-3-(trifluoromethyl)phenyl]carbamoyl}amino)propyl]sulfanyl}nonyl)sulfanyl]acetic acid (CP05)

CP05 was prepared from 24 according to the general procedure given for the hydrolysis of esters.

Glassy liquid that formed white solid over two days, yield 87%. ¹H NMR (500 MHz, DMSO-d₆): δ 9.07 (br, 1H), 8.06 (s, 1H), 7.56 (d, J=9.0 Hz, 1H), 7.52 (d, J=9.0 Hz, 1H), 6.50 (br, 1H), 3.17 (s, 2H), 3.14 (q, J=6.0 Hz, 2H), 2.54 (t, J=7.5 Hz, 2H), 2.50-2.40 (m, 4H), 1.67 (p, J=7.5 Hz, 2H), 1.55-1.45 (m, 4H), 1.35-1.25 (m, 4H), 1.25-1.20 (m, 6H).

### ({9-[(3-{[(4-Methylphenyl)carbamoyl]amino}propyl)sulfanyl]nonyl}sulfanyl)acetic acid (CP06)

CP06 was prepared from 25 according to the general procedure given for the hydrolysis of esters.

Glassy white solid which formed waxy solid over two days, yield 69%. ¹H NMR (500 MHz, CDCl₃): δ 7.90 (br, 1H), 7.16 (m, 2H), 7.09 (m, 2H), 5.18 (br, 1H), 3.34 (q, J=6.5 Hz, 2H), 3.23 (s, 2H), 2.68 (t, J=7.0 Hz, 2H), 2.52 (q, J=7.0 Hz, 4H), 2.34 (s, 3H), 1.81 (p, J=7.0 Hz, 2H), 1.70-1.60 (m, 4H), 1.45-1.35 (m, 4H), 1.35-1.30 (m, 6H).

### Biological Efficacy

### In Vitro Experiment

The compounds referred to in the experiments below correspond to the compound numbers as given in Table 1.

### Experimental

### Cell Culture:

Human MDA-MB-231, MDA-MB-468 and T-47D breast cancer cells were obtained from the American Type Culture Collection (ATCC; Manassas, Va., USA). Cells were plated on 96-well microplates at a density of 1×10⁴ cells per well for MTT, ATP, Caspase and JC-1 assays. Cells were maintained in Dulbecco's Modified Eagle's Medium (DMEM; Invitrogen, Carlsbad, Calif., USA) supplemented with 10% (w/v) fetal bovine serum (Hyclone, Salt Lake City, Utah, USA), 100 U/mL penicillin, and 100 μg/mL streptomycin (Invitrogen). All of the cells were incubated at 37° C. in a humidified atmosphere of 95% air and 5% CO₂. Confluent cells (80-90%) were washed in phosphate-buffered saline, harvested using Trypsin/EDTA, and counted using trypan blue exclusion in a Countess Automated Cell Counter (Invitrogen) before passaging or use in assays.

### MTT Assay:

MDA-MB-231, MDA-MB-468 and T-47D cells were seeded in 96 well plates (0.2 mL/well), treated with compound CP01 (1, 5, 10 or 20 μM for 24, 48 or 72 h). MTT (25 μL of 2.5 mg/mL solution) was added to each well for 2 h, after which MTT and media were removed. The blue formazan product formed from MTT by live cells was dissolved in dimethylsulphoxide (DMSO; 100 μL/well) and quantified spectrophotometrically at 540 nm in a multilabel counter. Data are means±SEM of at least three separate experiments conducted with internal triplicates.

### ATP Assay:

MDA-MB-231, MDA-MB-468 and T-47D cells were seeded in 96 well plates (0.2 mL/well), treated with compound CP01 (1, 5, 10 or 20 μM for 24, 48 or 72 h). Following treatments, plates were equilibrated at room temperature for 30 min, 100 μl of CellTiter-Glo® reagent was added (CellTiter-Glo® Assay; Promega, Annandale, NSW, Australia). Cells were lysed for 2 min, plates, incubated at room temperature for 10 min and luminescence was recorded in a multilabel reader. Data are means±SEM of at least three separate experiments conducted with internal triplicates.

### Caspase 3 Activity:

MDA-MB-231 cells were seeded in 96 well plates (0.2 mL/well), and were treated with compound CP01 for 48 h. Caspase 3 activity was quantified using the Caspase-Glo 3/7 assay kit according to the manufacturer's protocol (Promega; Annandale, NSW, Australia). Briefly, after 48 h treatment, fresh serum-free media was added to wells. Cells were equilibrated at room temperature for 30 min, caspase 3/7 substrate in lysis buffer was added and the luminescence was measured. Relative caspase 3/7 activity was calculated as [(luminescence in treatment—luminescence in control)/luminescence in control×100%].

### JC-1 Assay:

At the end of treatments, medium was removed and cells were incubated in darkness with 1 μg/mL JC-1 in serum-free DMEM for 30 min at 37° C. Following centrifugation (1250 rpm 5 min RT) and three washes times with PBS to remove dye, the fluorescence of JC-1 monomers and dimers were measured on a Fluoroskan Ascent FL microplate reader (Labsystems, Sweden) using filter pairs of 530 nm/590 nm (dimers; red, indicative of mitochondrial health) and 485/538 nm (monomers; green, indicative of mitochondrial damage). The red/green fluorescence ratio was determined relative to control (taken as 1.0).

### Matrigel Assay:

MDA-MB-231 cells were trypsinized and resuspended in serum-free DMEM media (3.5×10⁶ cells/mL). The cell suspension was mixed 1:1 with Matrigel solution (Bio Scientific; Kirrawee, NSW, Australia). Aliquots (20 μL containing 3.5×10⁴ cells) were placed into 6-well tissue-culture dishes to form well-defined droplets and incubated at 37° C. for 5 min to enable semi-solidification. Migration media was freshly made as DMEM containing 20% fetal bovine serum, epidermal growth factor 10 pg/mL, hydrocortisone 0.4 ng/mL, vascular endothelial growth factor (VEGF, 1 pg/mL), basic fibroblast growth factor (bFGF, 20 pg/mL), insulin-like growth factor-1 (40 pg/mL), ascorbic acid (2 ng/mL) and heparin (45 ng/mL). Compounds were added in migration media (3 mL/well) and plates were then incubated for 24 h. Cells that migrated out of droplets were scored using phase-contrast microscopy and digital image analysis (Olympus).

### Results

MTT reduction
MTT Reduction in Breast Cancer Cell Lines Treated with Compound CP01.

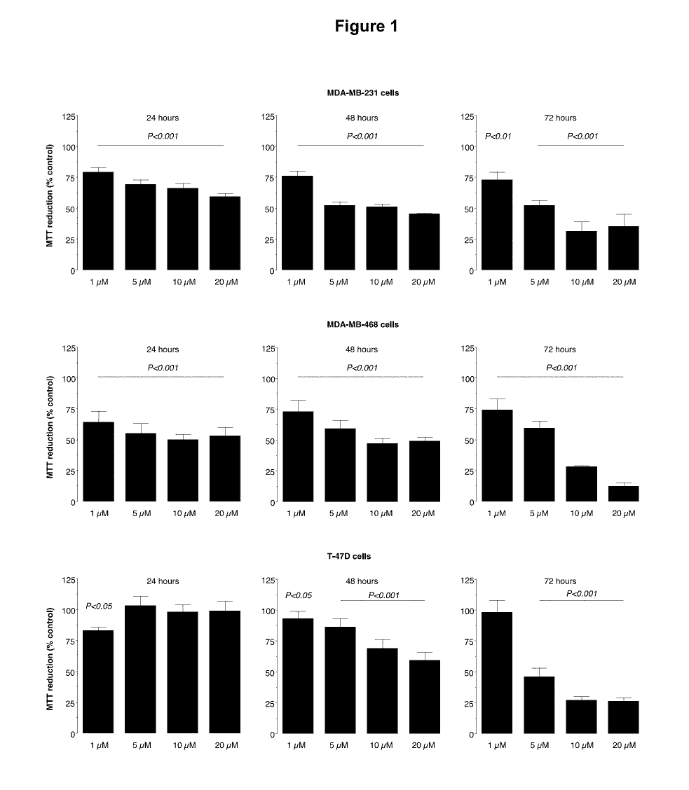

The effects of 24, 48 or 72 h treatments (1, 5, 10, 20 μM) treatment on MTT reduction were assessed in (top) MDA-MB-231, (middle) MDA-MB-468, and (bottom) T-47D cell lines (see FIG. 1). P-values were calculated by ANOVA and Fisher's PLSD testing. Data are mean±SEM of at least three independent experiments with three internal replicates.

### ATP Formation

ATP Formation in Breast Cancer Cell Lines Treated with Compound CP01.

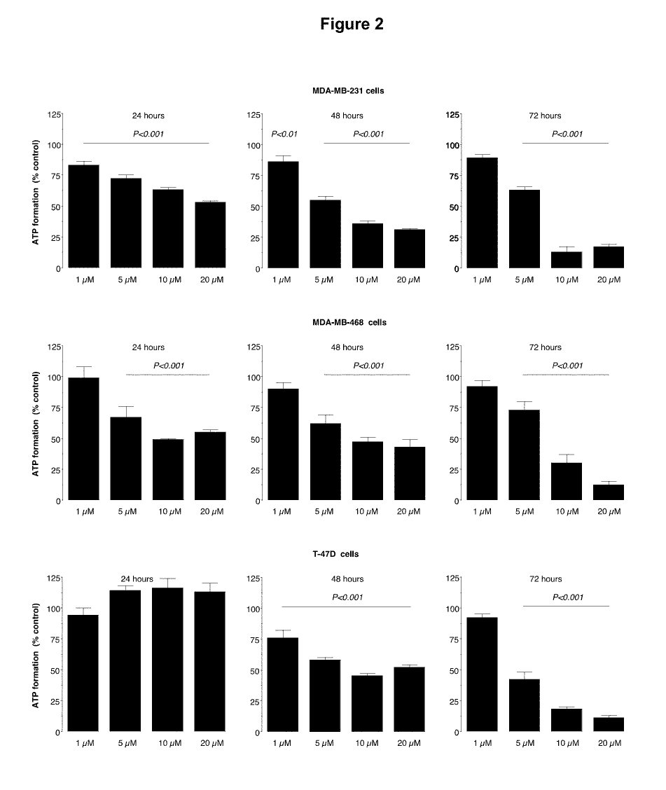

The effects of 24, 48 or 72 h treatments (1, 5, 10, 20 μM) treatment on MTT reduction were assessed in (top) MDA-MB-231, (middle) MDA-MB-468, and (bottom) T-47D cell lines (see FIG. 2). P-values were calculated by ANOVA and Fisher's PLSD testing. Data are mean±SEM of at least three independent experiments with three internal replicates.

### JC-1 and Caspase 3/7 Activity for Mitochondrial Integrity and Apoptosis.

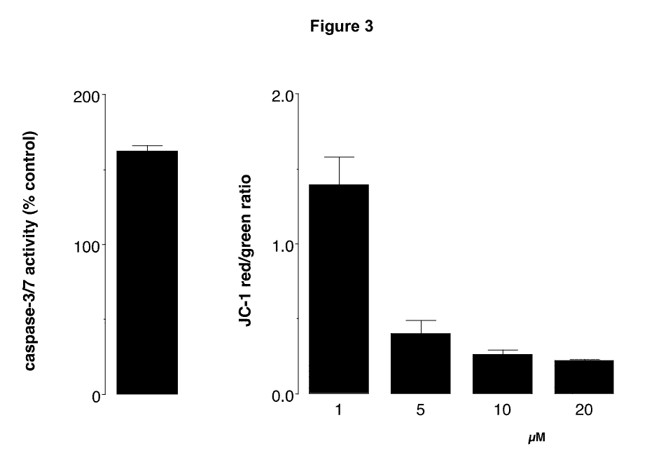

Left, Caspase 3/7 activation in MDA-MB-231 cells treated with CP01 (10 μM, 48 h), right, JC-1 red/green fluorescence ratio in MDA-MB-231 breast cancer cells treated with CP01 (1-20 μM, 4 h) (see FIG. 3). Data are as mean±SEM of at least three independent experiments with internal duplicates.

### Matrigel Migration Assay

Assays of the Migration of MDA-MB-231 Cells Out of Matrigel Droplets after treatment with (A) CP02 (B) CP04 and (C) CP06

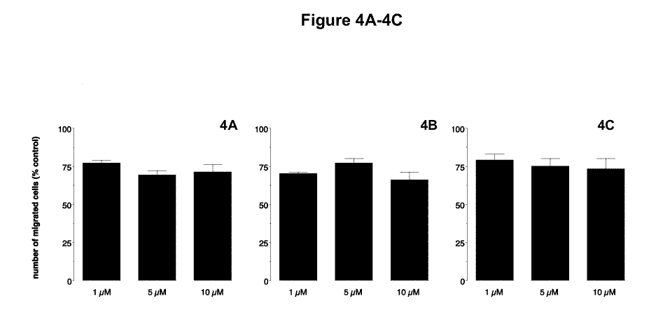

(see FIG. 4). Compounds were tested at concentrations of 1, 5 and 10 μM and are mean±SEM of at least three independent experiments; all decreases significant at least at P<0.01.

### In Vivo Experiments

Dose-response effects of compounds CP01 and CP03 on breast tumour growth in nude mice with human MDA-MB-231 breast cancer cell xenografts (subcutaneous injection in mammary fat pad)

### Experimental

### Mouse Species and Gender:

nu/nu Balb/c, female.

### Age:

5 weeks old when ordered, 6 weeks old when xenografted.

### Mouse Weight Range:

The weight range at the start of IP treatments was from 13.1 g to 18.1 g at Day 1 and increased to a range of 17.1 g to 21.3 g at Day 37.

### Tumour Cells and Xenografting:

human MDA-MB-231 cells, 4×10⁵ cells/100 μL (Matrigel:PBS 1:1)/mouse, subcutaneous injection into the third mammary fat pad.

### Groups and Mouse Number:

7 groups, 5 mice in each group. After the cell injection, 7 cages were randomly divided into 7 groups following the average mouse body weight of mice each cage.
Control
CP01-2.5 mg/kg
CP01-10 mg/kg
CP01-40 mg/kg
CP03-2.5 mg/kg
CP03-10 mg/kg
CP03-40 mg/kg

### Compound Stocks:

Solid CP01 or CP03 powder was dissolved in DMSO at 40° C. to a final concentration of 400 mg/mL.

TABLE 2
Compound preparation for IP injection

| | | | To make 1.0 mL injection | | |
|---|---|---|---|---|---|
| | | | corn oil solution with 4% | | |
| Dose | | Dilution of the | DMSO | | |
| (drug/mouse | Drug concentration | compound stock | corn | Compound | DMSO |
| body weight) | in corn oil (mg/mL) | (400 mg/mL DMSO) | oil (μL) | stock (μL) | (μL) |
| 40 mg/kg | 16 | 1:25 | 960 | 40 | 0 |
| 10 mg/kg | 4 | 1:100 | 960 | 10 | 30 |
| 2.5 mg/kg | 1 | 1:400 | 960 | 2.5 | 37.5 |
| 0 mg/kg | 0 | 0 | 960 | 0 | 40 |
| (Control) | | | | | |

### Treatment and Dosage:

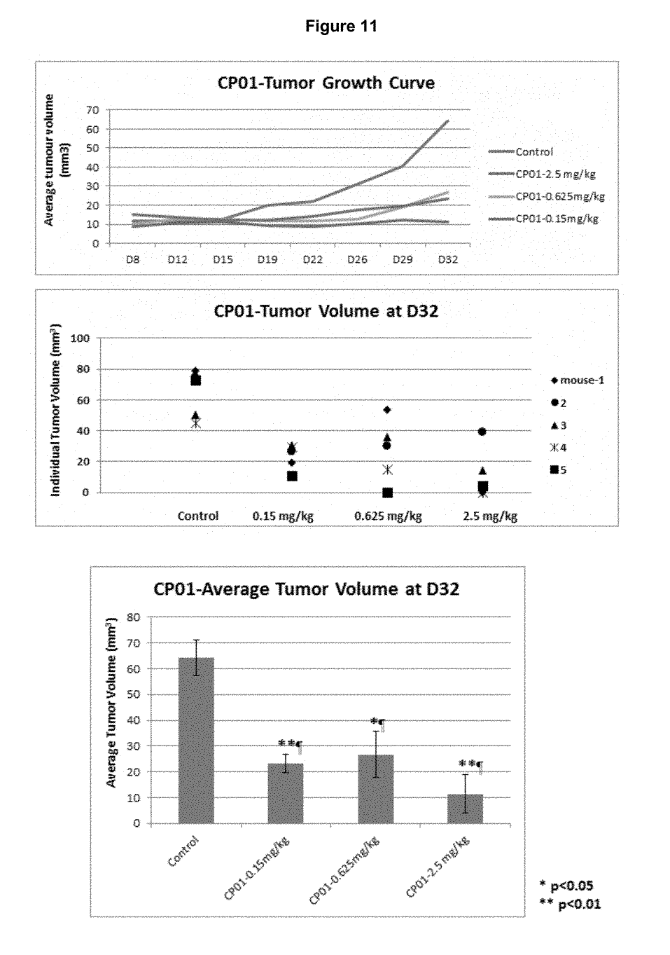

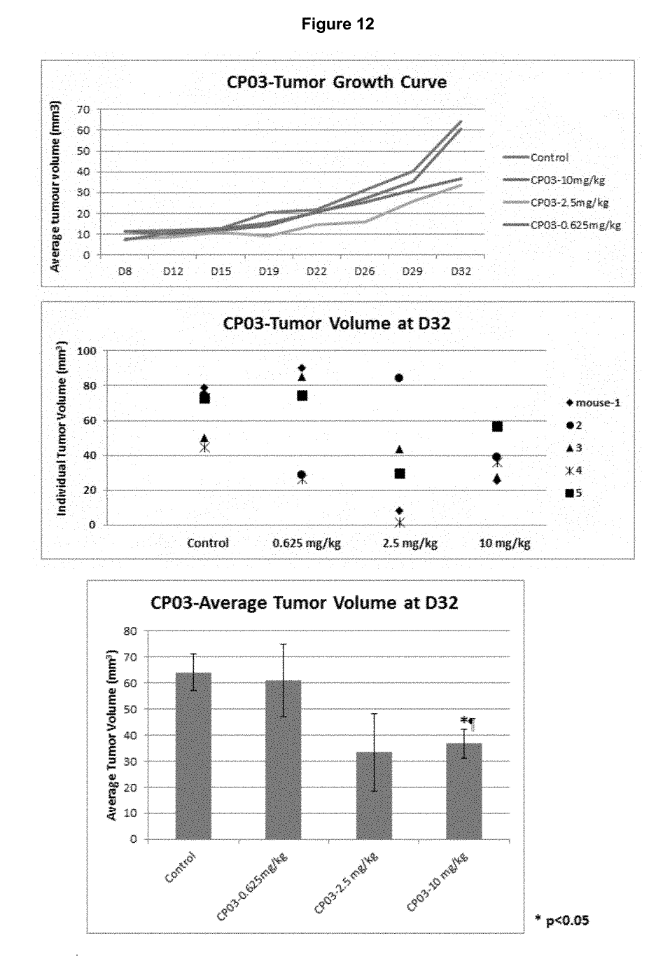

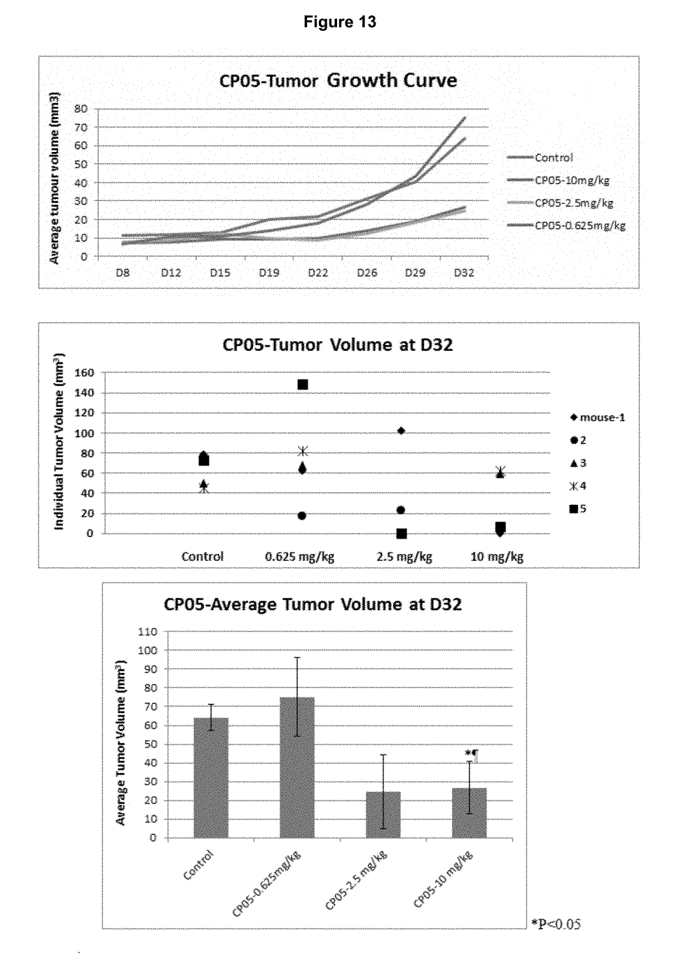

After 4 days of cancer cell xenografting, IP administration of compounds CP01 or CP03 was started (2.5, 10 and 40 mg/kg). Compounds were diluted in corn oil containing 4% DMSO. Injection volume was 50 μL each mouse with a 20-gram body weight. IP injections were administered once a day for six days per week for a total of 37 days in duration. In a further study (see below), different doses of CP01, CP03 and CP05 were administered to nude mice carrying MDA-MB-231 xenografts (FIGS. 11, 12 and 13, respectively). The endpoint in this experiment was tumour volume and the experiments were conducted for 32 days.

### Observation:

Body weights were recorded six days per week and tumour sizes were measured with a pair of caliper, every three to four days.

### Results

### Mouse Body Weight Growth in Control and the Treated Groups

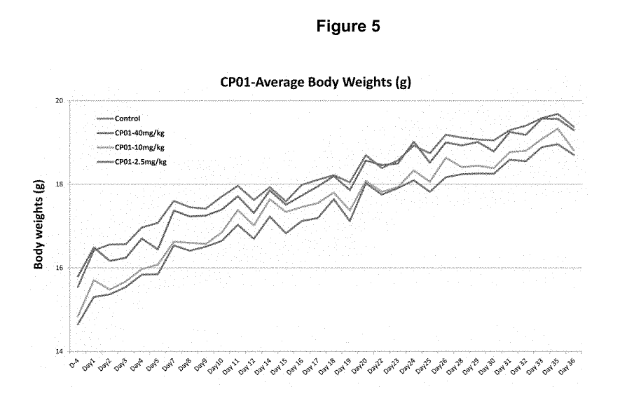

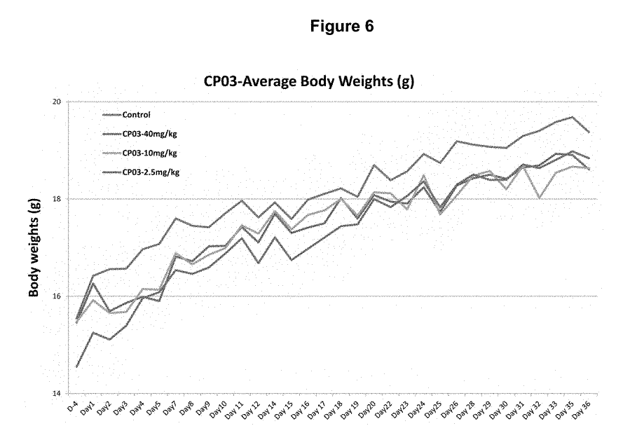

In groups treated with CP01 or CP03 (FIG. 5 and FIG. 6), after the initial IP injection there was a small decrease in body weight. Subsequently, all mice gained weight at a steady and similar rate during the whole experimental procedure. There was no significant difference observed in the treated mice compared with the mice in control group. Thus there was no evidence of toxicity.

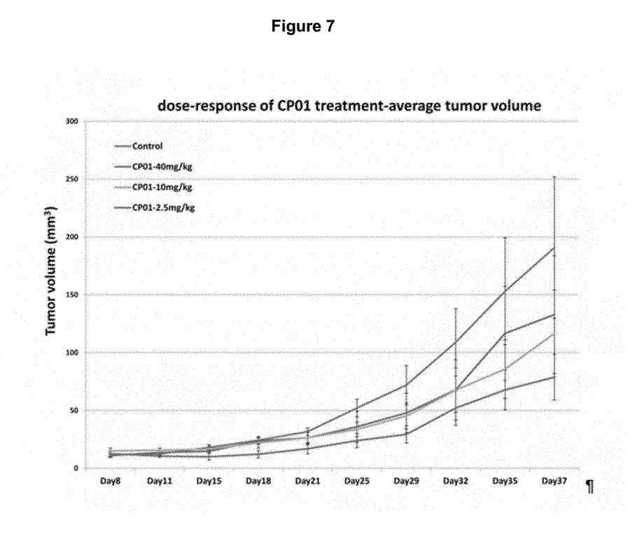

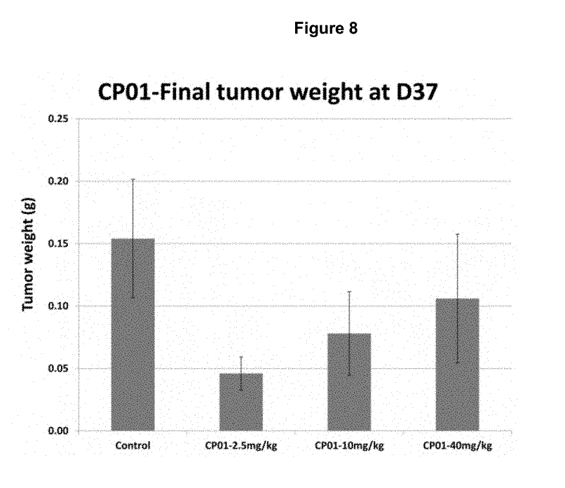

Effects of CP01 treatment on tumour growth (see Table 3 and FIG. 7) and on final tumour weight (see Table 4 and FIG. 8)

The suppression of tumour growth is indicated by a reduction of 59%, 39% and 30%, respectively, in tumour size at the doses of 2.5, 10 and 40 mg/kg.

TABLE 3
Average tumour volumes (mm³) in control and CP01-treated groups

| Group | Day 8 | Day 11 | Day 15 | Day 18 | Day 21 | Day 25 | Day 29 | Day 32 | Day 35 | Day 37 |
|---|---|---|---|---|---|---|---|---|---|---|
| Control | 11.8 ± 2.5 | 12.4 ± 1.5 | 18 ± 2.4 | 24.3 ± 3.2 | 31.7 ± 3.1 | 52.2 ± 7.7 | 71.9 ± 16.9 | 108.9 ± 29.1 | 153 ± 46.3 | 190.8 ± 61.2 |
| CP01- | 12.5 ± 1.9 | 10.5 ± 1 | 10 ± 2.9 | 12.2 ± 3.2 | 16.9 ± 4.4 | 23.9 ± 6.1 | 29.3 ± 7.4 | 52.1 ± 15 | 67.7 ± 17.1 | 78.7 ± 19.8 |
| 2.5 mg/kg | | | | | | | | | | |
| CP01- | 15 ± 2.4 | 15.6 ± 1.8 | 16.5 ± 2.8 | 22 ± 4.5 | 26.4 ± 4.5 | 33.6 ± 6.2 | 45.2 ± 11.2 | 67.5 ± 19.3 | 85.7 ± 25.3 | 116.5 ± 37.6 |
| 10 mg/kg | | | | | | | | | | |
| CP01- | 11 ± 1.3 | 13.6 ± 2 | 14.7 ± 0.7 | 23.6 ± 2.7 | 26.3 ± 6 | 35.9 ± 13.4 | 47.8 ± 17 | 67.8 ± 26 | 116.6 ± 40.7 | 132.8 ± 50.9 |
| 40 mg/kg | | | | | | | | | | |

Note:
Date is shown in mean ± SEM, n = 5

TABLE 4
Final tumour weight (g) at day 37 with CP01 treatment

| Group | at Day 37 |
|---|---|
| Control | 0.15 ± 0.05 |
| CP01-2.5 mg/kg | 0.05 ± 0.01 |
| CP01-10 mg/kg | 0.08 ± 0.03 |
| CP01-40 mg/kg | 0.11 ± 0.05 |

Note:
Date is shown in mean ± SEM, n = 5

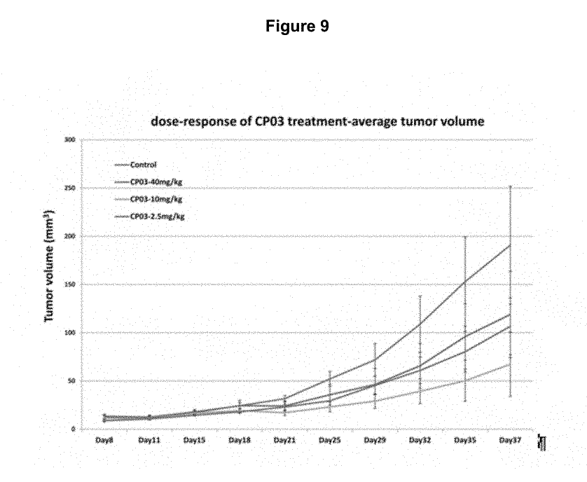

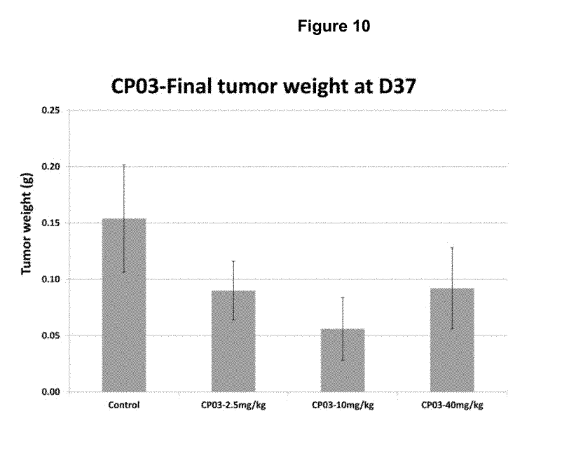

Effects of CP03 treatment on tumour growth (see Table 5 and FIG. 9) and on final tumour weight (see Table 6 and FIG. 10)

The suppression of tumour growth is indicated by a reduction of 44%, 65% and 38%, respectively, in tumour size at the doses of 2.5, 10 and 40 mg/kg.

TABLE 5
Average tumour volumes (mm³) in control and CP03-treated groups

| Group | Day 8 | Day 11 | Day 15 | Day 18 | Day 21 | Day 25 | Day 29 | Day 32 | Day 35 | Day 37 |
|---|---|---|---|---|---|---|---|---|---|---|
| Control | 11.8 ± 2.5 | 12.4 ± 1.5 | 18 ± 2.4 | 24.3 ± 3.2 | 31.7 ± 3.1 | 52.2 ± 7.7 | 71.9 ± 16.9 | 108.9 ± 29.1 | 153 ± 46.3 | 190.8 ± 61.2 |
| CP03- | 8.8 ± 1.4 | 10.7 ± 1.3 | 14.5 ± 0.9 | 18 ± 1.6 | 23 ± 2.8 | 29.5 ± 6 | 45.4 ± 9.5 | 60.9 ± 13.7 | 80.4 ± 21 | 106.7 ± 29.4 |
| 2.5 mg/kg | | | | | | | | | | |
| CP03- | 11.3 ± 2.9 | 10.9 ± 1.5 | 16.5 ± 2.8 | 19.3 ± 2.1 | 17.1 ± 2.9 | 23.1 ± 5 | 29 ± 7.5 | 39.3 ± 12.9 | 50.3 ± 21.2 | 67.3 ± 33.3 |
| 10 mg/kg | | | | | | | | | | |
| CP03- | 13.4 ± 2.3 | 12.5 ± 2 | 17.2 ± 1.9 | 24.4 ± 5.5 | 24 ± 5.2 | 35.7 ± 10.7 | 46.3 ± 16.7 | 65.7 ± 23 | 96.1 ± 33.8 | 118.9 ± 44.7 |
| 40 mg/kg | | | | | | | | | | |

Note:
Date is shown in mean ± SEM, n = 5

TABLE 6
Final tumour weight (g) at day 37 with CP03 treatment

| Group | at Day 37 |
|---|---|
| Control | 0.15 ± 0.05 |
| CP03-2.5 mg/kg | 0.09 ± 0.03 |
| CP03-10 mg/kg | 0.06 ± 0.03 |
| CP03-40 mg/kg | 0.09 ± 0.04 |

Note:
Date is shown in mean ± SEM, n = 5

These results show strong trends toward tumour suppression at all doses. The dose response shows a trend for greater activity at 2.5 (CP01) or 10 (CP03) mg/kg doses which may indicate some complexity in the dose responsiveness.

Effects of Lower Doses of CP01, CP03 and CP05 on Breast Tumour Growth in Balb/c Nude Mice with Human MDA MB 231 Breast Cancer Cell Xenografting

### Experimental

This experiment was carried out as described above, except that the dose range was different.

### Results

Treatment of breast tumours with compounds of the present invention has been shown to be effective in respect of the inhibition of breast tumour growth in the nude mice with human MDA-MB-231 breast cancer cell xenografting (t-test, p<0.05) (see FIGS. 11 to 13).

### Summary of In Vivo Experiments

The compounds of the present invention suppress breast tumour growth. The dose responses observed in these experiments are atypical. However, it should not be concluded that lower doses are necessarily more effective than higher doses. This is because responses to concentration differences are complex, due to the many pharmacokinetic and pharmacodynamic factors in play, and as such are not always linear.

### REFERENCES

Berquin I M, Edwards I J, Kridel S J, Chen Y Q. Polyunsaturated fatty acid metabolism in prostate cancer. Cancer Metastasis Rev. 2011, 30(3-4):295-309.
Chen J K, Falck J R, Reddy K M, Capdevila J, Harris R C. Epoxyeicosatrienoic acids and their sulfonimide derivatives stimulate tyrosine phosphorylation and induce mitogenesis in renal epithelial cells. J Biol Chem. 1998, 273(44):29254-61.
Inceoglue B, Schmelzer K R, Morisseau C, Jinks S L, Hammock B D. Soluble epoxide hydrolase inhibition reveals novel biological functions of epoxyeicosatrienoic acids (EETs). Prostaglandins Other Lipid Mediat. 2007, 82(1-4):42-9.

It will be understood that the invention disclosed and defined in this specification extends to all alternative combinations of two or more of the individual features mentioned or evident from the text or drawings. All of these different combinations constitute various alternative aspects of the invention.

1. A compound of formula (I): 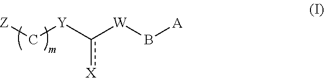
wherein
A is selected from OR¹, C(O)R¹, C(O)OR¹, C(O)NR¹R², OP(O)(OR¹)₂, C(O)OP(O)(OR)₂, P(OR¹)₃, C(O)OP(OR)₃, C(O)P(OR)₃, OS(O)(OR)₂, C(O)S(O)(OR)₂, OS(O)₂(OR¹), C(O)S(O)₂(OR¹), OSR¹, C(O)SR, OSR¹R², C(O)SR¹R², cycloalkyl, heterocycloalkyl and heteroaryl;
B is a hydrocarbon chain containing from 7 to 25 carbon atoms, wherein the hydrocarbon chain is saturated or unsaturated, branched or unbranched, and includes one or more heteroatoms selected from O, N and S;
W and Y are selected from CH₂, O and NR¹, wherein W may form a 5- or 6-membered cycloalkyl or heterocycloalkyl ring with X and B;
X is selected from CH₂, O, NR¹ and S;
C is CH₂;
m is 0, 1 or 2;
Z is selected from alkyl, heteroalkyl, alkenyl, alkynyl, cycloalkyl, heterocycloalkyl, aryl and heteroaryl, which groups are optionally substituted,
wherein R¹ and R² are independently selected from H, OH, alkyl, heteroalkyl, alkenyl, alkynyl, cycloalkyl, heterocycloalkyl, alkylcycloalkyl, heteroalkylcycloalkyl, aryl, heteroaryl, aralkyl and heteroaralkyl, which groups are optionally substituted,
or a pharmaceutically acceptable salt, solvate or hydrate thereof.

2. The compound of formula (I) according to claim 1, wherein A is C(O)OR¹.

3. The compound of formula (I) according to claim 2, wherein R¹ is H or alkyl.

4. The compound of formula (I) according to claim 3, wherein alkyl is methyl.

5. The compound of formula (I) according to claim 3, wherein alkyl is ethyl.

6. The compound of formula (I) according to any one of the preceding claims, wherein the hydrocarbon chain contains 13 carbon atoms.

7. The compound of formula (I) according to any one of claims 1 to 5, wherein the hydrocarbon chain contains 14 carbon atoms.

8. The compound of formula (I) according to any one of the preceding claims, wherein the hydrocarbon chain is saturated.

9. The compound of formula (I) according to any one of the preceding claims, wherein the hydrocarbon chain is unbranched.

10. The compound of formula (I) according to any one of the preceding claims, wherein the heteroatom(s) are in one or more of the 3-, 4-, 5-, 6-, 7-, 8-, 9-, 10-, 11-, 12-, and 13-positions along the hydrocarbon chain.

11. The compound of formula (I) according to claim 10, wherein the heteroatom(s) are at the 3- or 13-positions along the hydrocarbon chain.

12. The compound of formula (I) according to claim 10, wherein the heteroatom(s) are at the 3- and 13-positions along the hydrocarbon chain.

13. The compound of formula (I) according to any one of claims 1 to 11, wherein the hydrocarbon chain includes one S atom.

14. The compound of formula (I) according to any one of claims 1 to 10 or 12, wherein the hydrocarbon chain includes two S atoms.

15. The compound of formula (I) according to any one of claims 1 to 11, wherein the hydrocarbon chain includes one O atom.

16. The compound of formula (I) according to any one of claims 1 to 10 or 12, wherein the hydrocarbon chain includes two O atoms.

17. The compound of formula (I) according to any one of the preceding claims, wherein W and Y are both NH, X is O and the bond between X and the atom to which it is attached is a double bond.

18. The compound of formula (I) according to any one of the preceding claims, wherein Z is an aryl group.

19. The compound of formula (I) according to claim 18, wherein the aryl group is a phenyl group.

20. The compound of formula (I) according to claim 18 or 19, wherein the aryl group is substituted by an alkyl group or a halogen.

21. The compound of formula (I) according to claim 20, wherein the alkyl group is a methyl group.

22. The compound of formula (I) according to claim 20, wherein the halogen is fluorine or chlorine.

23. The compound of formula (I) according to claim 18 or 19, wherein the aryl group is substituted by one or more halogens, one or more alkyl groups, or combinations thereof.

24. The compound of formula (I) according to claim 23, wherein the aryl group is substituted by a halogen and an alkyl group.

25. The compound of formula (I) according to claim 24, wherein the halogen is chlorine.

26. The compound of formula (I) according to claim 24 or 25, wherein the alkyl group is substituted by one or more halogen atoms.

27. The compound of formula (I) according to claim 26, wherein the substituted alkyl group is CF₃.

28. A pharmaceutical composition including a therapeutically effective amount of a compound of formula (I) according to any one claims 1 to 27, or a mixture thereof, and one or more pharmaceutically acceptable excipients.

29. A method of treating a proliferative disorder including administering to a patient in need thereof a compound of formula (I) according to any one of claims 1 to 27, or a mixture thereof.

30. A method of treating a proliferative disorder including administering to a patient in need thereof a pharmaceutical composition according to claim 28.

31. The method according to claim 29 or 30, wherein the proliferative disorder is a metastatic cancer.

32. A method of inducing apoptosis in a cell, especially a cell undergoing cell division, the method including contacting the cell with a compound of formula (I) according to any one of claims 1 to 27, or a mixture thereof, or a composition according to claim 28.

33. A method of inhibiting cell migration, the method including contacting the cell with a compound of formula (I) according to any one of claims 1 to 27, or a mixture thereof, or a pharmaceutical composition according to claim 28.

34. Use of a compound of formula (I) according to any one of claims 1 to 27, or a mixture thereof, for the treatment of a proliferative disorder.

35. Use of a pharmaceutical composition according to claim 28 for the treatment of a proliferative disorder.

36. Use according to claim 34 or 35, wherein the proliferative disorder is a metastatic cancer.

37. Use of a compound of formula (I) according to any one of claims 1 to 27, or a mixture thereof, in the manufacture of a medicament for the treatment of a proliferative disorder.

38. Use according to claim 37, wherein the proliferative disorder is a metastatic cancer.
